(19) United States
(12) Patent Application Publication
(10) Pub. No.: US 2022/0326244 A1
(43) Pub. Date: Oct. 13, 2022

(54) METHODS FOR DIAGNOSIS, PROGNOSIS, AND TREATMENT OF CANCER

(71) Applicant: The Boardof Trustees of the Leland Stanford Junort University, Stanford, CA (US)

(72) Inventors: Wendy Fantl, Emeryville, CA (US); Veronica D. Gonzalez, Stanford, CA (US)

(21) Appl. No.: 17/640,283

(22) PCT Filed: Aug. 13, 2020
(86) PCT No.: PCT/US2020/046195

§ 371 Date: Mar. 3, 2022

Related U.S. Application Data
(60) Provisional application No. 62/897,775, filed on Sep. 9, 2019.

(51) Int. Cl. G01N 33/574; G01N 33/50; A61K 35/17

(52) U.S. Cl. CPC G01N 33/57449; G01N 33/5047; A61K 35/17; G01N 2333/70596

(57) ABSTRACT

Acquisition of CD9 protein on the cell surface of NK cells confers an immunosuppressive phenotype to the NK cells, making them less effective in immunotherapy. CD9 can be transferred from tumor cells to NK cells present in the tumor environment through the process of trogocyotosis. Methods of enhancing NK cell anti-tumor activity can include evaluating NK receptor ligand expression within the tumor microenvironment(s) for patients eligible to receive NK cell immunotherapy.

### CROSS REFERENCE

This application claims benefit of U.S. Provisional Patent Application No. 62/897,775 filed Sep. 9, 2019, which applications are incorporated herein by reference in their entirety.

### STATEMENT REGARDING FEDERALLY SPONSORED RESEARCH OR DEVELOPMENT

This invention was made with government support under contract OC110674 awarded by the Department of Defense. The government has certain rights in the invention.

### BACKGROUND

Tubo-ovarian high-grade serous cancer (HGSC) is the most lethal gynecologic malignancy mainly as the consequence of its advanced-stage diagnosis by which time it has metastasized to multiple sights making curative treatment challenging. Standard-of-care is surgical debulking and platinum-based chemotherapy with a 70 to 80% likelihood of recurrence within 5 years. Recently, the introduction of two new treatment modalities into the clinic has brought renewed hope to women with HGSC. One exploits the paradigm of synthetic lethality through the administration of small molecule poly (ADP-ribose) polymerase inhibitors (PARPi). These have been clinically approved for HGSCs harboring loss of function in BRCA1 or BRCA2 genes.

Immunotherapy, the second recently developed treatment modality, is aimed at restoring the ability of the patient's immune system to eradicate a tumor and is an approach mostly focused on reactivation of T lymphocytes. Although HGSC tumors show high frequencies of functionally exhausted T cells, and high levels of immune checkpoint proteins, such as PD-1, CTLA-4, LAG-3 and PD-L1, responses to immunotherapy for HGSC have been disappointing. Therefore, a deeper understanding of the cell types within the HGSC immune microenvironment could assist in identifying predictive mechanistic biomarkers to select patients likely to gain the most benefit from immunotherapy.

Since their discovery in 1975, natural killer (NK) cells have been recognized as innate lymphocytes that possess potent cytotoxic activity against tumors and virally infected cells. In addition, NK cells also produce an array of cytokines that regulate immune responses. NK cells are mechanistically distinct from T lymphocytes in that their cytotoxic activity occurs in an antigen-independent manner and without the need for prior sensitization. Instead, NK cell function results from the tightly regulated integration of intracellular signaling mediated by multiple germline surface receptors with both activating and inhibitory activities. In tumors these dual effector functions endow NK cells with roles in both immune surveillances to eradicate tumor cells and conversely with the creation of an immune tolerant microenvironment facilitating tumor progression.

### SUMMARY OF THE INVENTION

Compositions, methods, and kits are provided for enhancing cancer therapy involving natural killer (NK) cellular immunotherapy. It is shown herein that the acquisition of CD9 protein on the cell surface of NK cells confers an immunosuppressive phenotype to the NK cells, making them less effective in immunotherapy. The data indicate that CD9 can be transferred from tumor cells, including without limitation ovarian cancer cells, to NK cells present in the tumor environment through the process of trogocyotosis. In some embodiments of the invention the cancer is ovarian cancer. In some embodiments the cancer is ovarian serous cancer. In some embodiments the cancer is high-grade serous cancer (HGSC). In other embodiments, cancers that transfer CD9 to NK cells include colorectal carcinoma; breast, lung adenocarcinoma, non-small cell lung cancer, etc.

In one embodiment, a patient selected for treatment with NK cell immunotherapy is evaluated for expression of CD9 on the cancer cells prior to treatment. In some embodiments, the presence of a high level of cancer cells that express CD9 indicates a need to administer a CD9 blocking agent in combination with NK cell therapy, e.g. at least about 0.01% positive, at least about 0.1% positive, at least 1% positive, at least 10% positive, at least 20% positive, at least 30% positive, at least 50% positive, or more, of the cancer cell population is CD9⁺.

In some embodiments a patient is selected for NK cell immunotherapy where the cancer is determined to have a low level of CD9⁺ cells, for example less than about 50% CD9⁺ cells, less than about 40%, less than about 30%, less than about 20%, less than about 10% CD9⁺ cells, for example where an individual cancer is evaluated for expression of CD9 prior to treatment; or prior to stratification in a clinical trial, and a patient is selected for treatment accordingly.

Treatment with NK cell immunotherapy can include, without limitation, administration of an effective dose of an allogeneic or autologous population of NK cells. The NK cells can be expanded in vitro prior to administration. The NK cells can be differentiated from a progenitor cell population in vitro, e.g. from cord blood, hematopoietic stem cells, and the like. The NK cells can be an off the shelf cell product, including an NK cell line, e.g. NK 92 cells. The NK cells can be genetically modified prior to administration, e.g. by introduction of a CAR vector. Treatment with NK cell immunotherapy can also include administration of agents, e.g. antibodies, checkpoint inhibitors, BIKEs, TRIKEs, etc. that activate endogenous NK cells.

In treatment of cancer with NK cell immunotherapy, the immunotherapy may be provided in combination with an effective dose of an agent that blocks CD9, where the dose is effective to reduce inhibition of cytotoxic NK cell killing relative to administration without the CD9 blocking agent. Agents for this purpose include antibodies, peptides, soluble receptor, small molecules, and the like. Antibodies may specifically bind to CD9. Patients may be pre-treated with an effective dose of an agent that binds to CD9. An effective dose of an agent may be administered with the NK cells; or may be administered following NK cell administration.

In an alternative embodiment, the NK cells are treated with an agent that inhibits trogocytosis prior to administration to a patient. Agents for this purpose include, for example, concanavalin A, wortmannin, EDTA, nocodazole and cytochalasin D. In some embodiments the NK cells are treated with cytochalasin D prior to administration for treatment of a CD9⁺ cancer.

Methods of enhancing NK cell anti-tumor activity can include evaluating NK receptor ligand expression within the tumor microenvironment(s) for patients that have received NK cell immunotherapy. In one such embodiments, a peripheral blood test is used to monitor gain of CD9 expression by adoptively transferred NK cells, where a gain of CD9 is indicative that the NK cells are being down-regulated for cytotoxicity. In such embodiments, treatment is modified to reduce CD9 acquisition, or to provide alternative therapy. A blocking CD9 antibody can be administered before NK immunotherapy, or the NK cells can be treated to reduce marker acquisition by trogocytosis. Such biomarkers not only guide the selection of patients most likely to respond to NK immunotherapy, but can be used to monitor the durability of patient responsiveness.

In other embodiments methods are provided for determining the NK receptor ligand distribution in an ovarian cancer tumor to distinguish patients having ovarian cancer tumor cells carrying NK receptor ligands that activate NK cells, who are likely to benefit from NK cellular immunotherapy, from patients having ovarian cancer tumor cells carrying NK receptor ligands that inhibit NK cells, who are unlikely to benefit from NK cellular immunotherapy. In addition, markers of decidual-like NK cells indicating a poor prognosis for ovarian cancer patients are disclosed as well as methods of determining the frequency of decidual-like NK cells in a population of tumor infiltrating NK cells in order to identify individuals in need of treatment for ovarian cancer who are in need of more aggressive anti-cancer treatment. In some such embodiments the marker is CD9.

In one aspect, a prognostic method of predicting a poor prognosis for a patient having ovarian cancer and treating the patient for the ovarian cancer is provided, the method comprising: a) obtaining a sample of ovarian tumor tissue from the patient, wherein the ovarian tumor tissue comprises a population of infiltrating NK cells; b) measuring frequency of decidual-like NK cells in the population of infiltrating NK cells, wherein increased frequency of decidual-like NK cells compared to reference value ranges for a control population of NK cells indicates that the patient has a poor prognosis; and c) treating the patient with surgery, radiation therapy, chemotherapy, targeted therapy, anti-angiogenic therapy, or immunotherapy, or any combination thereof, if the patient is identified as having a poor prognosis.

In certain embodiments, measuring the frequency of decidual-like NK cells comprises detecting at least one NK cell expressing a CD9 marker, wherein expression of the CD9 marker indicates that the NK cell is a decidual-like NK cell.

In certain embodiments, the method further comprises detecting at least one decidual-like NK cell expressing a CD9 marker in combination with one or more additional markers selected from the group consisting of CD56 and chemokine receptor CXCR3.

In certain embodiments, the frequency of the decidual-like NK cells in the population of infiltrating NK cells is at least 29%. In certain embodiments, the frequency of the decidual-like NK cells in the population of infiltrating NK cells is at least 60%. In some embodiments, the frequency of the decidual-like NK cells in the population of infiltrating NK cells ranges from about 29% to about 70%, including any percentage within this range such as 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70%.

In certain embodiments, the method further comprises measuring levels of expression of one or more activating NK receptor ligands on cancerous cells in the sample of ovarian tumor tissue, wherein increased frequency of the decidual-like NK cells in combination with decreased levels of expression of one or more activating NK receptor ligands compared to the levels of expression of said NK receptor ligands on control ovarian cells indicates that the patient has a poor prognosis.

In certain embodiments, the method further comprises measuring levels of expression of one or more inhibitory NK receptor ligands on cancerous cells in the sample of ovarian tumor tissue, wherein increased frequency of the decidual-like NK cells in combination with increased levels of expression of one or more inhibitory NK receptor ligands compared to the levels of expression of said NK receptor ligands on control ovarian cells indicates that the patient has a poor prognosis.

In certain embodiments, the levels of NK receptor ligands are measured in ovarian cancer cells expressing E-cadherin (E tumor compartment), ovarian cancer cells coexpressing E-cadherin and vimentin (EV tumor compartment), and ovarian cancer cells expressing vimentin (V tumor compartment).

In certain embodiments, the method further comprises administering NK cellular immunotherapy to the patient if activating NK receptor ligands are detected on the ovarian cancer cells and increased levels of expression of the one or more inhibitory NK receptor ligands are not detected on the ovarian cancer cells. In some embodiments, the NK cellular immunotherapy comprises administration of one or more cytokines that activate NK cells to the patient, adoptive transfer of NK cells to the patient, or a combination thereof. In some embodiments, administering NK cellular immunotherapy comprises administering engineered NK cells comprising an NK activating receptor.

In certain embodiments, the method further comprises measuring frequency of NK cells in the population of infiltrating NK cells producing at least three cytokines selected from the group consisting of IL-8, IL-10, TNFα, GM-CSF and IFNγ, wherein increased frequency of the decidual-like NK cells in combination with decreased frequency of the NK cells producing at least three cytokines selected from the group consisting of IL-8, IL-10, TNFα, GM-CSF and IFNγ indicates that the patient has a poor prognosis.

In certain embodiments, the method further comprises measuring levels of perforin and granzyme B produced by the population of infiltrating NK cells, wherein increased frequency of the decidual-like NK cells in combination with decreased levels of the perforin and the granzyme B compared to the levels of the perforin and the levels of the granzyme B for a control population of NK cells indicate that the patient has a poor prognosis.

In certain embodiments, the frequency of decidual-like NK cells in the population of infiltrating NK cells in the sample of ovarian tumor tissue is measured by performing mass cytometry (cytometry by time of flight (CyTOF)), fluorescence based-flow cytometry, immunohistochemistry, immunofluorescence, CO-detection by indexing (CODEX), multiplexed ion beam imaging (MIBI), Cyclic immunofluorescence or other multi-parametric single cell analysis technology.

In another aspect, a method of predicting whether a patient having ovarian cancer will benefit from natural killer (NK) cellular immunotherapy and treating the patient for the ovarian cancer is provided, the method comprising: a) obtaining a sample of ovarian tumor tissue from the patient; b) measuring NK receptor ligand distribution on cancerous cells in the ovarian tumor tissue including CD9 expression, wherein detection of one or more activating NK receptor ligands indicates that the patient will benefit from NK cellular immunotherapy, and detection of one or more inhibitory NK receptor ligands indicates that the patient will not benefit from NK cellular immunotherapy; and c) administering NK cellular immunotherapy to the patient if the NK receptor ligand distribution indicates that the patient will benefit from NK cell immunotherapy.

In certain embodiments, the method is performed prior to treatment of the patient with the NK cellular immunotherapy or while the patient is undergoing immunotherapy.

In some embodiments, the NK cellular immunotherapy comprises administration of one or more cytokines that activate NK cells to the patient, adoptive transfer of NK cells to the patient, or a combination thereof. In some embodiments, administering NK cellular immunotherapy comprises administering engineered NK cells comprising an NK activating receptor.

In certain embodiments, the activating NK receptor ligands activate the NKG2D receptor. Exemplary activating NK receptor ligands include, without limitation, ULBP1, ULBP2, ULPBP3, ULPBP4, ULBP5, ULBP6, and MICA/B.

In certain embodiments, the method further comprises measuring frequency of NK cells in the population of infiltrating NK cells producing at least three cytokines selected from the group consisting of IL-8, IL-10, TNFα, GM-CSF and IFNγ, wherein decreased frequency of the NK cells producing at least three cytokines selected from the group consisting of IL-8, IL-10, TNFα, and IFNγ indicates that the patient will not benefit from NK cell immunotherapy.

In certain embodiments, the method further comprises measuring levels of perforin and granzyme B produced by the population of infiltrating NK cells, wherein decreased levels of the perforin and the granzyme B compared to the levels of the perforin and the levels of the granzyme B for a control population of NK cells indicate that the patient will not benefit from NK cell immunotherapy.

In certain embodiments, the methods described herein are performed on an ovarian cancer patient having high-grade serous ovarian cancer. In certain embodiments, the sample of ovarian tumor tissue is a biopsy or surgical specimen.

### BRIEF DESCRIPTION OF THE DRAWINGS

FIGS. 1A-1D: HGSC tumor and EV cell frequencies correlate with a dl-NK cell phenotype. (A) Hierarchically organized heat-map showing pairwise Spearman correlations between total tumor and total EV cell frequencies with specific immune cell clusters. Enlarged portions of the heat map (right-hand side) depict positive (red) and negative (blue) rₛ correlations, respectively with dl-NK cell clusters. Positive correlations were observed between total frequencies of tumor and EV cells with dl-NK cell clusters (squares) and two T cell clusters (circles). Clusters with decidual-like features (triangles) do not correlate with tumor and EV cells but are present in all tumors. (B) Phenotype of dl-NK cell clusters depicted by their protein expression patterns (C) dl-NK cells manually gated from the CD45+ CD66− immune cell infiltrate or from the total NK cell population positively correlated with total EV cell frequency. (D) dl-NK cells manually gated from the CD45+ CD66− immune cell infiltrate negatively correlated with a sub-group of vimentin clusters.

FIGS. 2A-2D: Expression patterns of NK receptor ligands in newly diagnosed HGSC tumors. Single cell force directed layouts (FDLs) are composites of twelve HGSC tumors. 10,000 cells sampled from each of 56 X-shift tumor cell clusters are color-coded by expression of: (A) E-cadherin and vimentin, (EV clusters co-expressing E-cadherin and vimentin are encircled and labeled 1 to 7 (15)), NKG2D receptor ligands and ADAM proteases (B) nectin-family ligands (C) HLA-ABC and HLA-E inhibitory ligands and tumor associated antigens CA125, mesothelin and HE4. (D) Box and whisker plots show distribution of expression levels for each NK receptor ligand and ADAM proteases 10 and 17 across the 12 HGSC tumors. p-values: **≤0.01, ***≤0.001, ***≤0.0001, for overall ANOVA comparing NK receptor ligand expression across E, EV and V compartments. Medians and interquartile ranges are shown.

FIGS. 3A-3C: Combinatorial diversity for NK receptor ligands within E, EV and V HGSC tumor compartments. (A) Boolean logic was used to determine the combinatorial diversity of 12 NK receptor ligands and ADAM 10 and 17 proteases expressed by tumor cells. Each NK receptor ligand combination is a row (left hand side). The heat map (right hand side) shows the frequency of tumor cells within the E, EV, and V compartments for 12 tumor samples (columns) that express each ligand combination. Rows were ranked based on the highest (top) to lowest total cell frequency (bottom). (B) Venn diagram depicts number of distinct and overlapping NK receptor ligand combinations across E, EV and V compartments. (C) The Gini-Simpson's inverse index of diversity was significantly greater for the E (p=0.05) and EV (p=0.007) versus V tumor compartments across all HGSC tumors. Medians and inter quartile ranges are shown.

FIG. 4. Responses to carboplatin across E, EV and V HGSC cell lines. OVCAR4 (E), Kuramochi (EV) and TYK-nu (V) cell lines exposed to vehicle or carboplatin at 0.5 or 1 μg/ml for 1 week were processed for CyTOF with the tumor NK receptor ligand/ADAM antibody panel. The parent population is defined as viable single cells negative for cisplatin and cPARP. The plots show frequencies of HGSC cells expressing activating and inhibitor NK receptor ligands (X-axis) gated out of the parent population (Y-axis). Plots show the mean of triplicates with standard deviations. p-values: *≤0.05, **≤0.005, for overall ANOVA.

FIGS. 5A-5F: Trogocytosis from HGSC to NK-92 cell lines. HGSC and NK-92 cell lines were cocultured for 6 h at an effector (NK-92):target (OVCAR4) ratio of 1:1 unless otherwise indicated (Materials and Methods). (A) Frequency of CD9 expression in NK-92 cells post coculture with OVCAR4, Kuramochi and TYK-nu with and without transwell, respectively. Mean with standard deviations are shown (n=4). Exemplary 2D flow plots showing induction of CD9-expressing NK-92 cells after coculture. (B) Histograms confirm lack of extra- and intracellular CD9 protein expression in the NK-92 cell line but high levels in OVCAR4 cell line both cell(C) RT-PCR of sorted CD9+ and CD9− NK-92 cells after coculture with OVCAR4 cells. Data are presented as number of copies (upper plots) or fold change gene expression after coculture compared to respective monocultures (lower plots). (D) Pre-incubation of NK-92 cells with cytochalasin D (10 μg), a trogocytosis inhibitor, before coculture with HGSC cell line results in partial inhibition of trogocytosis (E) Transfer of membrane fragments with CD9 from OVCAR4 cells labeled with PKH67, onto NK-92 cells, after coculture (PKH67 upper histograms and CD9 lower histograms). (F) Visualization of trogocytosis by microscopy. OVCAR4 cells and NK-92 cells were labeled with PKH67 and PKH26, respectively. After coculture for 3 h cells were fixed in paraformaldehyde and stained with antibodies against CD45 and CD9. Cells were imaged in all channels on a Keyence BZ-X800 microscope. Images for cells grown in monoculture are shown at 20× and for coculture 60×. Images were enhanced for brightness and contrast to optimize the printed image.

FIGS. 6A-6D: Determining trogocytosis of CD9 from non-HGSC cells. (A) Eleven HGSC and 15 non-HGSC tumor cell lines were screened by CyTOF for CD9 expression. (B) The bar chart shows cells ranked by their level of CD9 expression. Cell lines were selected for coculture with NK-92 cells; HGSC E, EV and V cell lines (magenta), non HGSC cell lines with high levels of CD9 (green) and non-HGSC cell lines with lower levels of CD9 (yellow). (C) Representative flow plots showing the frequency of non-HGSC tumor cells that acquired CD9 (D) Preincubation of NK-92 cells with cytochalasin D (10 μM) results in partial inhibition of trogocytosis.

FIGS. 7A-7B: Intracellular cytokine production by CD9+ and CD9− NK-92. HGSC and NK-92 cells were cocultured at a 1:1 ratio for 6 h with PMA/ionomycin or vehicle control and brefeldin A/monensin for 4 h. There were two positive controls; NK-92 cells grown in monoculture −/+PMA and coculture between the K562 cell line (HLA-null erythroleukemic) with NK-92 cells (Methods). Cells were processed for CyTOF and stained with the NK cell antibody panel. CD9+ and CD9− cells were manually gated from the CD45+ cell population. Plots show the mean of triplicates with standard deviations. Student's two-tailed t-tests determined statistically significant functional differences between CD9+ and CD9− NK-92 cells. (A) Frequency of CD9+ and CD9− cells producing each cytokine as indicated. (B) Levels (raw median counts) of each cytokine produced in CD9+ and CD9− NK-92 cells. p-values: *≤0.01, **≤0.001, ***≤0.0001, ****≤0.00001.

FIGS. 8A-8C. NK-92 cell cytotoxicity toward HGSC cell lines. HGSC cell lines (OVCAR4 (E), Kuramochi (EV) and TYK-nu (V) were assayed for their susceptibility to NK-92 mediated cytotoxicity as measured by the calcein release assay after coculture for 4 h (n=3). (A) NK-92 cells have reduced cytotoxicity toward HGSC cell lines compared to the control K562 cell line, at the target:effector ratios shown. (B) performing the calcein release assay in the presence of a CD9 blocking antibody significantly restores NK-92 cytotoxicity compared to an isotype control. Data are shown for quadruplets performed with two antibody concentrations and different target:effector cell ratios. Statistical significance determined with two tailed t test: *p≤0.05, **p≤0.01, p≤0.001. (C) FACS-sorted CD9+ NK-92 after coculture have a reduced cytotoxicity function compared to their CD9− counterpart grown in monoculture.

### DETAILED DESCRIPTION OF THE EMBODIMENTS

Compositions, methods, and kits are provided for prognosis of ovarian cancer patients and predicting responsiveness to treatment with natural killer (NK) cellular immunotherapy. In particular, methods are provided for determining the NK receptor ligand distribution in an ovarian cancer tumor to distinguish patients having ovarian cancer tumor cells carrying NK receptor ligands that activate NK cells, who are likely to benefit from NK cellular immunotherapy, from patients having ovarian cancer tumor cells carrying NK receptor ligands that inhibit NK cells, who are unlikely to benefit from NK cellular immunotherapy. In addition, markers of decidual-like NK cells indicating a poor prognosis for ovarian cancer patients are disclosed as well as methods of determining the frequency of decidual-like NK cells in a population of tumor infiltrating NK cells in order to identify individuals in need of treatment for ovarian cancer who are in need of more aggressive anti-cancer treatment.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a biomarker” includes a plurality of such biomarkers and reference to “the polypeptide” includes reference to one or more polypeptides and equivalents thereof, e.g. peptides or proteins known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

“CD9” is a member of the transmembrane 4 superfamily also known as the tetraspanin family. It is a cell surface glycoprotein that consists of four transmembrane regions and has two extracellular loops that contain disulfide bonds which are conserved throughout the tetraspanin family. Palmitoylation sites that allows CD9 to interact with lipids and other proteins. Their distinct palmitoylation sites allow them to organize on the membrane into tetraspanin-enriched microdomains (TEM). These TEMs are thought to play a role in many cellular processes including exosome biogenesis.

CD9 can modulate cell adhesion and migration. It has a varying role in different types of cancers. The over expression of CD9 was shown to decrease metastasis in certain types of melanoma, breast, lung, pancreas and colon carcinomas. However in other studies, CD9 has been shown to increase migration or be highly expressed in metastatic cancers in various cell lines such as lung cancer, scirrhous-type gastric cancer, hepatocellular carcinoma, acute lymphoblastic leukemia, and breast cancer.

CD9 has been shown to interact with CD117; CD29, CD46, CD49c, CD81, PTGFRN, TSPAN4, CD63, ADAM17, and CD81. The reference sequence for human CD9 may be accessed at Genbank, NP_001317241 and NP_001760.

Anti-CD9 agent. As used herein, the term “anti-CD9 agent” refers to any agent that reduces the presence of CD9 on an NK cell. Non-limiting examples of suitable anti-CD9 reagents reduce the trogocytosis transfer of CD9 from cancer cells to NK cells. The efficacy of a suitable anti-CD9 agent can be assessed by assaying the agent. In an exemplary assay, target cells are incubated in the presence or absence of the candidate agent. An agent for use in the methods of the invention will up-regulate NK cell mediated killing, e.g. by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) compared to the level in the absence of the agent.

Markers for upregulated NK cell mediated killing include, for example, expression of granzyme B, perforin and MIP1β. NK cell degranulation can be measured with CD107a. VEGF levels are high with active NK cells, as is TNFα, GM-CSF and IFNγ. Down regulated NK cells, in contrast, express IL-8 and IL-10.

Anti-CD9 antibodies. In some embodiments, a subject anti-CD9 agent is an antibody that specifically binds CD9 (i.e., an anti-CD9 antibody). The antibody may reduce the transfer of CD9 to NK cells from cancer cells. In some embodiments, a suitable anti-CD9 antibody does not activate CD9 upon binding. Suitable anti-CD9 antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies from, for example camelids such as llama or camel; and variants thereof.

Anti-CD9 antibodies are known and used in the art, for example see Santos et al. J Cell Mol Med (2019) 23(6):4408-4421, Anti-human CD9 antibody Fab fragment impairs the internalization of extracellular vesicles and the nuclear transfer of their cargo proteins; WO2017119811A1 anti-CD9 antibody, each herein specifically incorporated by reference.

In some embodiments, a therapeutically effective dose of an anti-CD9 agent leads to sustained serum levels of about 40 μg/ml or more (e.g, about 50 ug/ml or more, about 60 ug/ml or more, about 75 ug/ml or more, about 100 ug/ml or more, about 125 ug/ml or more, or about 150 ug/ml or more). In some embodiments, a therapeutically effective dose leads to sustained serum levels of that range from about 40 μg/ml to about 300 ug/ml (e.g, from about 40 ug/ml to about 250 ug/ml, from about 40 ug/ml to about 200 ug/ml, from about 40 ug/ml to about 150 ug/ml, from about 40 ug/ml to about 100 ug/ml, from about 50 ug/ml to about 300 ug/ml, from about 50 ug/ml to about 250 ug/ml, from about 50 ug/ml to about 200 ug/ml, from about 50 ug/ml to about 150 ug/ml, from about 75 ug/ml to about 300 ug/ml from about 75 ug/ml to about 250 ug/ml, from about 75 ug/ml to about 200 ug/ml, from about 75 ug/ml to about 150 ug/ml, from about 100 ug/ml to about 300 ug/ml, from about 100 ug/ml to about 250 ug/ml, or from about 100 ug/ml to about 200 ug/ml). In some embodiments, a therapeutically effective dose for treating solid tumors leads to sustained serum levels of about 100 μg/ml or more (e.g., sustained serum levels that range from about 100 ug/ml to about 200 ug/ml).

Natural Killer (NK) Cell therapy. Natural killer (NK) cells, are important mediators of cancer immunosurveillance. NK cells are a heterogeneous population. In humans there are many subtypes of NK cells, which can vary by expression levels of markers including CD56, CD16, and the like, e.g. IFNγ-producing CD56^{hi}CD16⁺; cytotoxic CD56^{lo}CD16^{hi}; decidual-like NK cells CD56⁺CD9⁺CXCR3⁺KIR⁺CD3⁻CD16⁻; etc.

Signals from activating and inhibitory receptors tune the steady-state responsiveness of NK cells. Inhibitory receptors, such as killer-cell immunoglobulin-like receptors (KIRs), deliver negative signals that prevent NK cell autoreactivity. KIRs and other inhibitory receptors recognize MHC I molecules. Activating receptors, including NKG2D, provide activating signals upon binding to stress-induced ligands on target cells. NK cells sense and respond to changes in the repertoire of molecules expressed on the surface of healthy cells during cellular transformation. This positions NK cells as important sentinels against cancer and as prime targets for cancer immunotherapy.

Chemotherapy and radiotherapy mediate their effects, at least partially, via the immune system. Both chemo- and radiotherapy induce cellular stress in tumor cells, leading to upregulation of NK-activating ligands, release of damage-associated molecular patterns (DAMPs), and induction of immunogenic cell death. Through different mechanisms, genotoxic agents, HSP90 inhibitors, histone deacetylase (HDAC) inhibitors, glycogen synthase kinase 3 (GSK-3) inhibitors, and proteasome inhibitors can all increase tumor surface expression of NK-activating ligands. Several chemotherapeutics downregulate the NK inhibitory ligands such as MHC I on tumors.

NK therapy can be approached by administration of an effective dose of NK cells to a patient of either unmodified or modified cells, or by activating endogenous NK cells. Allogeneic NK cells and autologous NK cells have been explored for cancer immunotherapy. NK cells can be isolated and ex vivo expanded from the peripheral blood of patients. Approaches include different combinations of activating cytokines (IL-2, IL-12, IL-15, IL-18) and the use of feeder cells to supply important factors during ex vivo expansion. Additional strategies have been investigated to provide readily available banks of NK cells for patients for off the shelf use.

For example, the human cell line NK92 has been clinically investigated as an allogeneic NK therapeutic. NK92 (Neukoplast™) has been infused in multiple doses of, for example, 1×10⁹ cells/m² dose, 3×10⁹ cells/m² dose, 5×10⁹ cells/m² dose, and the like.

NK cells can be differentiated from stem cells, both induced pluripotent stem cells (iPSCs) and those obtained from umbilical cord blood. iPSC-derived NK cells have been shown to have high cytotoxicity against tumors of various origin, both in vitro and in vivo and clinical trials have commenced using expanded cord blood-derived NK cells. NK cells derived from peripheral blood iPSCs show low KIR expression and a capacity to perform both cellular cytotoxicity and antibody-dependent cellular cytotoxicity (ADCC) against cancer cell lines in vitro.

CAR-NK cells. A promising avenue in adoptive NK therapy is the use of chimeric antigen receptors (CARs). A CAR, usually encoded in a lentiviral construct, consists of three main domains: an extracellular antigen-targeting domain (ectodomain), a transmembrane region, and one or more intracellular signaling domains. Specificity for targets is conferred by the ectodomain, which is reactive against a tumor-specific or tumor-associated antigen (e.g., CD19, CD20, CD22, Her2, ROR1). CARs are now being used to potentiate NK antitumor activity.

Antibody therapy also provides an off-the-shelf approach to activating NK cells in vivo. In addition to traditional approaches that rely on tumor-binding monoclonal antibodies to activate NK cells via ADCC, bispecific killer cell engagers (BiKEs) are small molecules consisting of two scFvs with different specificity complexed together through flexible linkers. One scFv targets a tumor antigen (e.g., CD19, CD20, CD33), while the other is specific for an NK cell receptor (CD16). This effectively brings the cancer and NK cells together, facilitating the formation of an immunological synapse and allowing NK cells to specifically and effectively execute their cytolytic functions.

BiKEs' primary target has been CD16, as it potently induces NK activation without additional costimulation. BiKEs were able to redirect autologous NK cells against tumor cells and overcome the immunosuppression prevalent in these conditions. Additional scFvs, such as tri- and tetra-specific killer cell engagers (TriKEs and TetraKEs), can further potentiate therapeutic benefits by targeting more tumor antigens or adding IL-15 into the engager construct.

NK cells express many checkpoint receptors, some of which have been targeted by cancer immunotherapy. The majority of KIRs are inhibitory and recognize HLA molecules. To replicate missing self recognition, the humanized antagonistic antibody lirilumab targeting inhibitory KIRs (KIR2DLs1-3 and KIR2DSs1-2) is in clinical development, although lack of efficacy was associated with loss of NK cell responsiveness and loss of surface KIR2D expression via trogocytosis. CD94/NKG2A is a heterodimeric inhibitory receptor expressed on NK and T cells that recognizes peptide-bound HLA-E. In both solid tumors and hematological malignancies, HLA-E is upregulated to evade recognition by NK and T cells, and its expression is associated with poor prognosis. Blocking NKG2A, for example with Monalizumab, showed enhanced antitumor immunity by both T and NK cells in various tumor models. The inhibitory receptor TIM-3 is constitutively expressed on human NK cells and is upregulated in response to cytokine stimulation. Like PD-1, TIM-3 expression can mark NK cells that produce IFN-γ and release cytotoxic granules as well as NK cells with an exhausted phenotype.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide that is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BiTE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016)/nt J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen (e.g., biomarker) refers to a binding reaction that is determinative of the presence of the antigen in a heterogeneous population of proteins and other biologics. Thus, under designated immunoassay conditions, the specified antibodies bind to a particular antigen at least two times over the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of ovarian cancer sample(s) was used and/or how it was used, the technique used to assay the sample, the results of the assay (e.g., the level of the biomarker as measured and/or the fold-change of a biomarker level over time or in a post-treatment assay compared to a pre-treatment assay), the assessment as to whether the individual is determined to have decidual-like NKs (i.e., immune tolerant to tumor cells), a recommendation for treatment (e.g., NK cellular immunotherapy if decidual-like NKs are detected in sample of ovarian tumor tissue), and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

Cancers expressing CD9 are of particular interest.

In some embodiments the cancer is ovarian cancer. Ovarian cancer is often fatal because it is usually advanced when diagnosed. Symptoms are usually absent in early stages and nonspecific in advanced stages. Evaluation usually includes ultrasonography, CT or MRI, and measurement of tumor markers (eg, cancer antigen 125). Diagnosis is by histologic analysis. Staging is surgical. Treatment requires hysterectomy, bilateral salpingo-oophorectomy, excision of as much involved tissue as possible (cytoreduction), and, unless cancer is localized, chemotherapy. Probably 5 to 10% of ovarian cancer cases are related to mutations in the autosomal dominant BRCA gene, which is associated with a 50 to 85% lifetime risk of developing breast cancer. Women with BRCA1 mutations have a 20 to 40% lifetime risk of developing ovarian cancer. Mutations in several other genes, including TP53, PTEN, STK11/LKB1, CDH1, CHEK2, RAD51, BRIP1, PALB2, ATM, MLH1, and MSH2, have been associated with hereditary breast and/or ovarian cancer.

Ovarian cancers are histologically diverse. At least 80% of ovarian cancers originate in the epithelium; 75% of these cancers are serous cystadenocarcinoma, and about 10% are invasive mucinous carcinoma. About 20% of ovarian cancers originate in primary ovarian germ cells or in sex cord and stromal cells or are metastases to the ovary (most commonly, from the breast or GI tract). Germ cell cancers usually occur in women <30.

A “reference level” or “reference value” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in ovarian cancer samples (e.g. mass cytometry (CYTOF), immunoassays (e.g., ELISA), mass spectrometry (e.g., LC-MS, GC-MS), tandem mass spectrometry, immunohistochemistry, CODEX, etc.), where the levels of biomarkers may differ based on the specific technique that is used.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

Obtaining and assaying a sample. The term “assaying” is used herein to include the physical steps of manipulating a sample of ovarian tumor tissue to generate data related to the sample. As will be readily understood by one of ordinary skill in the art, a sample of ovarian tumor tissue must be “obtained” prior to assaying the sample. Thus, the term “assaying” implies that the sample has been obtained. The terms “obtained” or “obtaining” as used herein encompass the act of receiving an extracted or isolated sample of ovarian tumor tissue. For example, a testing facility can “obtain” a sample of ovarian tumor tissue in the mail (or via delivery, etc.) prior to assaying the sample. In some such cases, the sample of ovarian tumor tissue was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a sample of ovarian tumor tissue from a subject. Accordingly, a sample of ovarian tumor tissue can be isolated from a subject (and thus “obtained”) by the same person or same entity that subsequently assays the sample. When a sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a sample.

In some embodiments, the step of obtaining comprises the step of isolating a sample of ovarian tumor tissue (e.g., a pre-treatment sample, a post-treatment sample, etc.). Methods and protocols for isolating samples of ovarian tumor tissue (e.g., a biopsy, a surgical specimen, etc.) will be known to one of ordinary skill in the art and any convenient method may be used to isolate a sample of ovarian tumor tissue.

It will be understood by one of ordinary skill in the art that in some cases, it is convenient to wait until multiple samples (e.g., a pre-treatment sample and a post-treatment sample) have been obtained prior to assaying the samples. Accordingly, in some cases an isolated sample (e.g., a pre-treatment sample, a post-treatment sample, etc.) is stored until all appropriate samples have been obtained. One of ordinary skill in the art will understand how to appropriately store a variety of different types samples of ovarian tumor tissue and any convenient method of storage may be used (e.g., refrigeration) that is appropriate for the particular sample. In some embodiments, a pre-treatment sample is assayed prior to obtaining a post-treatment sample. In some cases, a pre-treatment sample and a post-treatment sample are assayed in parallel. In some cases, multiple different post-treatment samples and/or a pre-treatment sample are assayed in parallel. In some cases, samples are processed immediately or as soon as possible after they are obtained.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI) or mass units as determined from mass cytometry measurements), or can be expressed as an absolute value with defined units (e.g., number of mRNA transcripts, number of protein molecules, concentration of protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple samples from the same individual (e.g., samples taken at different points in time from the same individual). This is because the units cancel when calculating a fold-change (i.e., determining a ratio) in the expression level from one sample to the next (e.g., samples taken at different points in time from the same individual).

For measuring RNA levels, the amount or level of an RNA in the sample is determined, e.g., the level of an mRNA. In some instances, the expression level of one or more additional RNAs may also be measured, and the level of biomarker expression compared to the level of the one or more additional RNAs to provide a normalized value for the biomarker expression level. Any convenient protocol for evaluating RNA levels may be employed wherein the level of one or more RNAs in the assayed sample is determined.

For measuring protein levels, the amount or level of a protein in the sample is determined. In some cases, the protein comprises a post-translational modification (e.g., phosphorylation, glycosylation) associated with regulation of activity of the protein such as by a signaling cascade, wherein the modified protein is the biomarker, and the amount of the modified protein is therefore measured. In some embodiments, an extracellular protein level is measured. For example, in some cases, the protein (i.e., polypeptide) being measured is a secreted protein (e.g., cytokine) and the concentration can therefore be measured in fluid. In some embodiments, concentration is a relative value measured by comparing the level of one protein relative to another protein. In other embodiments the concentration is an absolute measurement of weight/volume or weight/weight.

In some instances, the concentration of one or more additional proteins may also be measured, and biomarker concentration compared to the level of the one or more additional proteins to provide a normalized value for the biomarker concentration. Any convenient protocol for evaluating protein levels may be employed wherein the level of one or more proteins in the assayed sample is determined.

While a variety of different manners of assaying for protein levels are known to one of ordinary skill in the art and any convenient method may be used, one representative and convenient type of protocol for assaying protein levels is ELISA, an antibody-based method. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed. Representative exemplary methods include but are not limited to antibody-based methods (e.g., immunofluorescence assay, radioimmunoassay, immunoprecipitation, Western blotting, proteomic arrays, xMAP microsphere technology (e.g., Luminex technology), immunohistochemistry, flow cytometry, mass cytometry, CYTOF, and the like) as well as non-antibody-based methods (e.g., mass spectrometry or tandem mass spectrometry).

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent, e.g. an infusion of engineered NK cells, etc., sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

The NK cells and CD9 blocking agent may be used alone or in combination with other therapeutic intervention such as radiotherapy, chemotherapy, immunosuppressant and immunomodulatory therapies.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Upid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting PD-L1 and those targeting CTLA-4 such as ipilimumab and those targeting KIR proteins and other NK cell specific agents.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

### Cell Compositions

In some embodiments a NK cell composition is provided in combination with a CD9 blocking agent. The cell can be provided in a unit dose for therapy, and can be allogeneic, autologous, etc. with respect to an intended recipient. Methods may include a step of obtaining desired cells, e.g., NK cells, hematopoietic stem cells, etc., which may be isolated from a biological sample, or may be derived in vitro from a source of progenitor cells. The cells are optionally transduced or transfected with a vector of interest, e.g. CAR construct, which step may be performed in any suitable culture medium. For example, cells may be collected from a patient, modified and/or expanded ex vivo, and reintroduced into the subject. The cells collected from the subject may be collected from any convenient and appropriate source, including e.g., peripheral blood (e.g., the subject's peripheral blood), a biopsy (e.g., a biopsy from the subject), and the like.

Where the use of autologous cells is not desirable, e.g. where a patient has insufficient NK cells for modification, where there is insufficient time to expand autologous cells, etc., allogeneic cells may be used, e.g. NK cells or stem cells from a healthy donor, or from an NK cell line such as the NK92 cells.

Cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow drip), intraarterial, intraperitoneal, intrathecal or subcutaneous administration.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### High Grade Serous Ovarian Tumor Cells Modulate NK Cells to Create an Immune Tolerant Microenvironment

Tubo-ovarian high-grade serous cancer (HGSC) exhibits a complex tumor-immune microenvironment with significant frequencies of exhausted T cells but is unresponsive to immunotherapy. Using mass cytometry (CyTOF) we investigated whether this lack of response could be accounted for by specific sub-populations of intra-tumoral T and/or NK cells. Analysis of newly diagnosed chemo-naïve HGSC tumors revealed that frequencies of decidual-like (dl)-NK cell sub-populations (CD56+CD9+CXCR3+KIR+CD3−CD16−) correlated positively with the total abundance of both tumor cells and transitioning epithelial-mesenchymal cells. Decidual NK cells confer immune tolerance at the fetal-maternal interface and provide a source of pro-angiogenic factors to vascularize the placenta. By analogy, we sought to discover how HGSC tumor cells manipulate dl-NK cell function toward an immune tolerant state. Investigation of NK receptor ligands within newly diagnosed HGSC tumors identified tumor cells with different combinatorial expression patterns for both activating and inhibitory NK receptors. This study revealed different combinatorial NK receptor ligand expression patterns between three tumor compartments: i) epithelial (E), expressing E-cadherin, ii) transitioning epithelial-mesenchymal (EV), expressing E-cadherin and vimentin and iii) with a more inhibitory phenotype, metastatic (V) expressing vimentin. Moreover, treatment with carboplatin of HGSC cell lines, which phenocopied E, EV and V tumor cells, generated an overall more inhibitory NK ligand receptor phenotype thereby identifying a previously unrecognized mechanism of carboplatin resistance. Notably an NK cell line (NK-92) when co-cultured with HGSC cell lines acquired CD9 from the ovarian tumor cells by trogocytosis with the consequent acquisition of immunosuppressive properties as reflected by both a reduction in immunogenic cytokines and cytotoxicity. Critically, CD9 is highly expressed in primary HGSC tumors. Since the NK-92 cell line and other sources of NK cells are in clinical development for adoptive immunotherapy the data from this study has critical relevance.

CyTOF analysis of cocultures between ovarian tumor cell lines and the clinically relevant NK-92 cell line provides mechanistic insight into how HGSC tumor cells could direct NK cell function to create an immunosuppressive environment that favors tumor survival. NK cells are now at the center of a variety of immunotherapeutic approaches to exploit their tumor cell killing activity. The single cell data from this study identifies critical and unappreciated mechanisms by which HGSC cells are able to subvert the killing activity of NK cells, providing urgent consideration when optimizing NK cell-based immunotherapy.

### Results

CyTOF analysis and generation of T and NK cell clusters from HGSC tumors. Here we report our analysis of the immune cell infiltrate from the/above HGSC tumors with a CyTOF antibody panel designed to characterize T and NK cell subtypes. All steps for quality control and CyTOF processing of barcoded samples were as previously described (Materials and Methods). For each tumor, the immune cell infiltrate was gated out as CD45+ CD66− from the viable immune cell population (Materials and Methods). The resultant T and NK immune single cell data sets were then combined and subjected to unsupervised analysis using X-shift clustering. Using 25 surface markers delineating T and NK cell subpopulations, 52 X-shift cell clusters (calculated for an optimal “k” of 30 nearest neighbors) were generated for subsequent analysis.

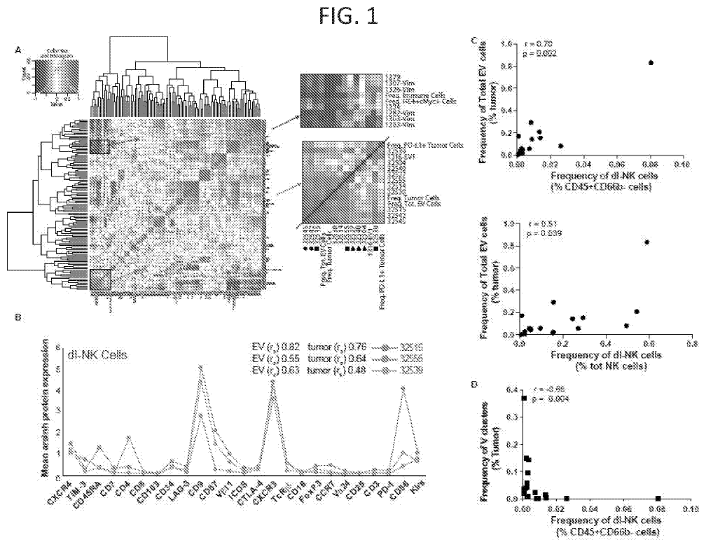

Correlation analysis between tumor and immune cell clusters from HGSC tumors. In order to understand the interactions between tumor and immune cells we applied a network approach that computes correlations between the frequencies of the cell phenotypes. The resultant pair-wise Spearman correlation coefficients (rₛ) were displayed on a hierarchically clustered heat-map (FIG. 1A). For each HGSC tumor, pairwise correlations included the following parameters i) Cell frequencies for all 52 T and NK cell clusters, ii) Cell frequencies for all 56 tumor cell clusters iii) Total tumor cell frequency iv) Total E cell frequency v) Total V cell frequency vi) Total EV cell frequency vii) other features described in our previous publication.

In the first part of our analysis we determined whether any T or NK cell clusters were correlated with abundance of all tumor cells or abundance of E, EV or V cells. We noted the presence of five immune cell clusters that correlated with both total tumor and EV cell abundance (Spearman's correlation coefficient, rₛ>0.5) (FIG. 1A). Three of these immune cell clusters (32515, 32539 and 32555) were negative for CD3 and CD16 expression but positive for expression of CD56, CD9, CXCR3 and killer immunoglobulin-like receptors (KIRs), a phenotype that resembles decidual NK cells (FIG. 1B). Notably, among the thousands of NK cell phenotypes, CD9 expression is exclusive to decidual NK (dNK) cell subsets. The combined cell frequency of these three decidual-like NK (dl-NK) clusters ranged from 1.3%-28% across the 17 tumors. Furthermore, correlations determined with manually gated dl-NK cells and EV tumor cells were consistent with the unsupervised X-shift analysis (FIG. 1C and FIG. 9).

A further two positively correlated immune cell clusters (32542 and 32545) had a T-cell phenotype (CD3+ with mutually exclusive expression of CD4 and CD8) but also shared phenotypic properties with decidual NK cells. They had high levels of CD56, CXCR3 and CD9 and low levels of the invariant T cell receptor Vα24-Vβ11 suggesting that these cells could have NKT-like functions (FIG. 10A). They were present in the HGSC immune cell infiltrate with a frequency of 0.1-3.9%.

An additional three dl-NK cell clusters (32527, 32504 and 32540) were found that did not correlate with either tumor or EV cell abundance (FIG. 1A and FIG. 10B). Cluster 32527 correlated with dl-NK cell clusters 32555 and 32539 (FIG. 1B). Clusters 32504 and 32540 did not correlate with any tumor features but were present in all tumors with a combined frequency range of 14%-86% of the immune cell infiltrate. To visualize the relationships between all T and NK immune cell clusters, a minimum spanning tree was generated computationally. This revealed that clusters 32504 and 32540 were phenotypically similar to both dl-NK and T cell clusters (FIG. 11). Given their high frequencies, these two clusters could act as source from which dl-NK cells are derived signifying previously unappreciated phenotypic plasticity between these NK and T cell types.

Of the eight immune cell clusters described above, five correlated negatively (rₛ>−0.6) with vimentin cell abundance (FIG. 1D). These data are consistent with published reports describing an inverse relationship between metastases and NK cells within immune infiltrates. Decidual NK cells play a critical role in the first trimester of pregnancy by conferring immune tolerance toward the hemi-allogeneic fetus and facilitating placental growth. The identification of dl-NK cells in HGSC led us to hypothesize that the same features of NK-mediated immune tolerance for the maintenance of pregnancy could be subverted for HGSC tumor maintenance and progression.

NK receptor ligand expression across newly diagnosed HGSC tumors. Having previously identified the E, EV and V intra-tumor cell compartments, each representing different stages of disease progression we wished to determine how these compartments modulated NK cell function toward an immune-tolerant state. We therefore analyzed 935,563 single intact cells prepared from 12 newly diagnosed late stage HGSC tumors (Materials and Methods). The goal was to compare frequencies of HGSC tumor cells that were expressing NK receptor ligands within the E, EV and V tumor compartments. Our modified HGSC CyTOF panel now included antibodies against twelve NK receptor ligands and two ADAM proteases (a disintegrin and metalloproteinase). The panel included antibodies against the following NK receptor ligands and ADAM proteases: i) ULBP1, ULBP2/5/6, ULPBP3, ULPBP4 and MICA/B that bind to the NKG2D activating NK receptor, ii) ADAM10 and ADAM17 proteases involved in NK ligand and NK receptor shedding, iii) nectin-like ligands, CD111, CD112, CD113, CD155 and nectin-4. These bind the activating NK receptor, CD226 (also known as DNAM1) and the inhibitory NK receptors, T cell immune-receptor with immunoglobulin and ITIM domains (TIGIT) and CD96 (also known as TACTILE), iv) Human leukocyte antigen (HLA) class I molecules, A, B and C that bind to inhibitory killer immunoglobulin-like receptors (KIR) and, v) HLA-E, a non-classical HLA class I molecule, that binds to the NK inhibitory receptor heterodimer CD94/NKG2A with greater affinity than to the activating CD94/NKG2C heterodimer.

NK receptor ligand expression levels across tumor cell compartments. In order to visualize the expression levels of the NK receptor ligands on tumor cells, CyTOF datasets for each of the twelve HGSOC samples were manually gated as previously described (CD45−, CD31−, FAP−) thereby excluding immune, angiogenic and stromal cells. The resultant single cell data files were combined and clustered using the X-shift algorithm. Tumor cells were clustered as before with the tumor markers E-cadherin, CD73, CD61, CD90, CD151, CD49f, CD133, ROR1, CD10, CD13, endoglin, CD24, CD44, MUC16 (CA125), mesothelin, vimentin, and HE4.

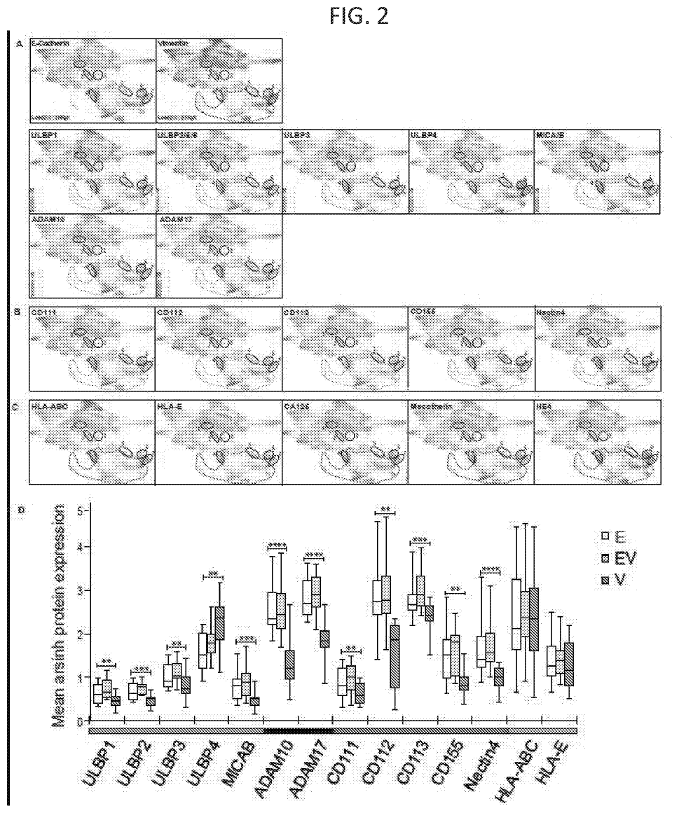

Spatial relationships between tumor cells expressing NK receptor ligands within the 56 X-shift tumor cell clusters were visualized by force directed layouts (FDLs). 10,000 single cells were computationally sampled from each X-shift cluster and each cell was connected on a 10-nearest-neighbor graph (Materials and Methods). This graph was subjected to a force-directed layout that placed groups of phenotypically related cells adjacent to one another (FIG. 2, A-C, Materials and Methods). Single cell sub-sampling with generation of FDLs was repeated three times with comparable results. The resultant FDLs were composites of tumor cells from all twelve samples and corroborated the presence of the E and V compartments and the EV compartment comprised of seven clusters as previously reported (FIG. 2A, upper two panels with V and EV cells encircled). In general, the FDLs revealed that receptor ligands, for both activating and inhibitory NK receptors, were expressed at variable levels in all three compartments within pockets of tumor cells, rather than evenly interspersed throughout all the tumor cells (FIG. 2, A-C).

Previous studies have demonstrated that upregulation of ligands for the NKG2D activating NK receptor is a major mechanism by which NK cells are able to detect and eradicate tumor cells. Our data revealed that, aside from ULPBP4, the highest levels of all the NKG2D receptor ligands were found in the E and EV HGSC tumor compartments with minimal levels in the V compartment (FIG. 2A). Overall, these data are consistent with an immune-surveillance role for NK cells within the E and EV tumor cell compartments. The high levels of ULBP4 expression throughout the V cell compartment are an exception consistent with a recent study describing ULBP4 as a functional outlier within the ULBP family.

For the proteases ADAM10 and ADAM17, discrete pockets of cells with high expression levels were observed in the E compartment and in the EV1 transitioning tumor cell subset. Some pockets of ADAM-expressing tumor cells co-localized with NKG2D ligands suggesting an attempt by tumor cells to nullify NK cell killing activity by promoting NK ligand shedding. In contrast to the NKG2D ligands that bind only activating receptors, the nectin family of ligands bind both activating and inhibitory NK receptors. These ligands also exhibited variable expression patterns with pockets of E tumor cells co-expressing high levels of CD112, CD113 and CD155, while EV2 tumor cells co-expressed high levels of CD112 and CD113, and similarly for EV3 tumor cells and EV5 CD113, CD155 and nectin 4 were significant (FIG. 2B).

HLA-A, B, C and E primarily engage NK cell inhibitory receptors to provide self-tolerance for healthy cells. These ligands were also co-expressed in pockets of cells at varying levels in all three tumor compartments (FIG. 2C, lower panels). To note, in contrast to their role in inhibiting tumor destruction by NK cells, recognition of MHC class I molecules expressed by tumor cells, targets them for destruction by cytotoxic CD8 T lymphocytes (CTLs). This occurs in an antigen-dependent mechanism whereby MHC I molecules present peptide fragments from tumor associated antigens (TAAs) to CTLs. Well-established TAAs in HGSC are MUC16, mesothelin and HE4. For the most part TAAs and HLAs were mutually exclusive (FIG. 2C). These data suggest that HGSC cells may have evolved dual escape mechanisms from the killing activity of both NK and CD8 T cells.

Quantifying expression levels of NK receptor ligands. In order to quantify how expression levels of NK receptor ligands varied across individual HGSC samples we generated box and whisker plots for each NK receptor ligand and the ADAMs in the E, EV and V tumor compartments across all the tumors (FIG. 2D). Statistical analysis revealed that for the most part, expression levels for the NK receptor ligands and ADAMs did not differ significantly between the E and EV compartments. However, expression levels for these proteins, aside from ULBP4, were all statistically lower in the V compartment. These data suggest that metastatic V cells have largely “escaped” immune surveillance by reducing their expression levels of NK receptor activating ligands.

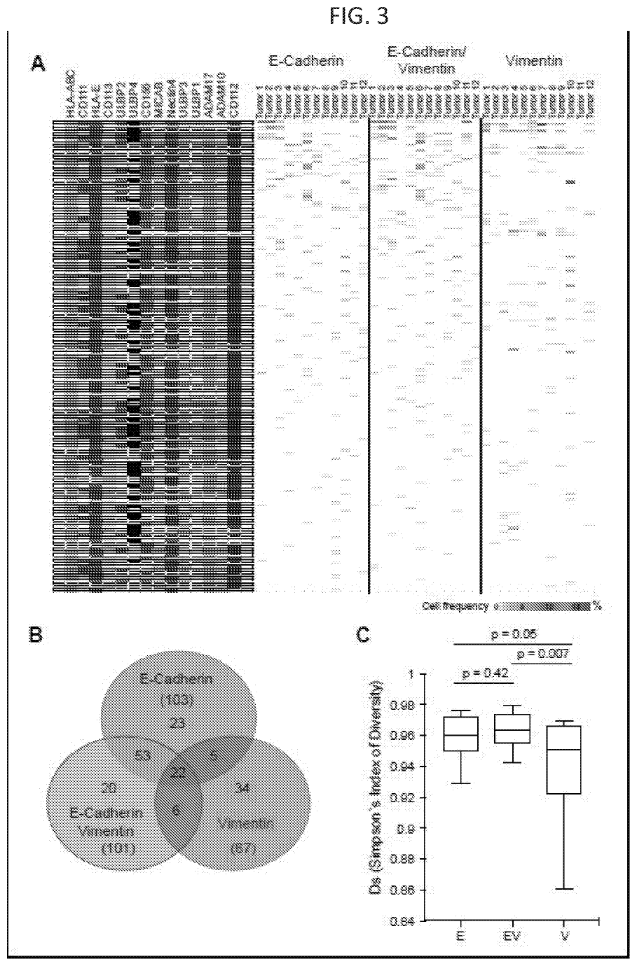

Quantifying the combinatorial diversity of NK receptor ligand expression. As discussed, the combinatorial expression patterns of a large repertoire of activating and inhibitory receptors endow NK cells with a high degree of phenotypic and functional diversity. In their turn, these receptors are regulated by a correspondingly complex repertoire of activating and/or inhibitory ligands present on tumor cells. This necessarily implies that tumor cells have the potential to play a major role in orchestrating NK cell function to shape the tumor immune microenvironment. In order to determine potential differences within the immune microenvironment of the E, EV and V tumor compartments, we applied Boolean analysis to measure the frequency of cells with distinct combinatorial expression patterns for the twelve NK receptor ligands and two ADAM proteases. We assessed 2¹⁴ or 16,348 NK receptor ligand combinations and compared the frequency of tumor cells harboring specific NK receptor ligand combinations (in the E, EV and V tumor compartments) (FIG. 3A, Materials and Methods). Using a threshold cell frequency of >1% for cells in any compartment in any sample expressing an NK receptor ligand/ADAM protease combination there were 163 NK receptor ligand combinations expressed by tumor cells in the 12 HGSCs (FIG. 3A, left-hand side with ligand combinations in rows).

Boolean analysis demonstrated that cells in the E and EV tumor compartments had greater combinatorial diversity for NK receptor ligands/ADAMs (103 and 101 phenotypes respectively) than the V tumor compartment (67 phenotypes) (FIG. 3B). There were more shared phenotypes between the E and EV compartments (53) than between the E and V (five) and EV and V (six) compartments (FIG. 3B). These data suggest differential regulation of the immune microenvironment orchestrated by the tumor cells in each of the three compartments.

To quantify further the NK receptor ligand combinatorial diversity in the different tumor cell compartments we calculated the Simpson's index of diversity. (FIG. 3C). This index is often used in ecology to quantify the biodiversity within a natural habitat and was recently applied to NK and ovarian tumor CyTOF datasets. When applied here, the Simpson's index of diversity was significantly higher in the E and EV compartments compared to the V tumor compartment (FIG. 3C) consistent with the greater number of NK receptor ligand combinations in the E and EV compartments compared to the V compartment.

NK receptor ligand expression across HGSC cell lines. In their genetic analysis of ovarian cancer cell lines Domcke et al. presented a list of ovarian cell lines ranked by the concordance of their genetics to resected HGSC tumors, with the goal of providing more reliable in vitro models of HGSC. In order to determine how the phenotypes of these cell lines compared with HGSC tumor cells, we analyzed thirteen of the highest ranked HGSC cell lines with our CyTOF tumor antibody panel modified with antibodies against NK receptor ligands and ADAMs. Data analysis performed with X-shift clustering and FDL visualization, revealed HGSC cell lines that phenocopied E, EV and V tumor compartments based on expression levels of E-cadherin and vimentin (Figure S4A) in addition to other measured tumor markers.

Examination of the NK receptor ligand/ADAM expression levels across the E, V and EV cell lines revealed patterns that, although not identical, were comparable to their E, V and EV counterparts in HGSCs (FIG. 12). For example, the NKG2D activating ligands were expressed primarily in E and EV cell lines but at very low levels in V cell lines (FIGS. 12B and E). For in vitro experiments we subsequently selected three HGSC cell lines, OVCAR4, Kuramochi and TYK-nu, that represent E, EV and V HGSC tumor cells, respectively.

Changes in NK receptor ligand expression levels in response to carboplatin. It is well established that activation of the DNA damage response with genotoxic agents increases the expression of ligands for NKG2D and DNAM1 thereby making a “stressed” cell more susceptible to NK cell killing. We therefore exposed the three HGSC cell lines to carboplatin, a genotoxic agent that is part of the standard-of-care regimen for women with HGSC. After one week, the HGSC cell lines were processed for CyTOF using the tumor/NK receptor ligand antibody panel (Table 12).

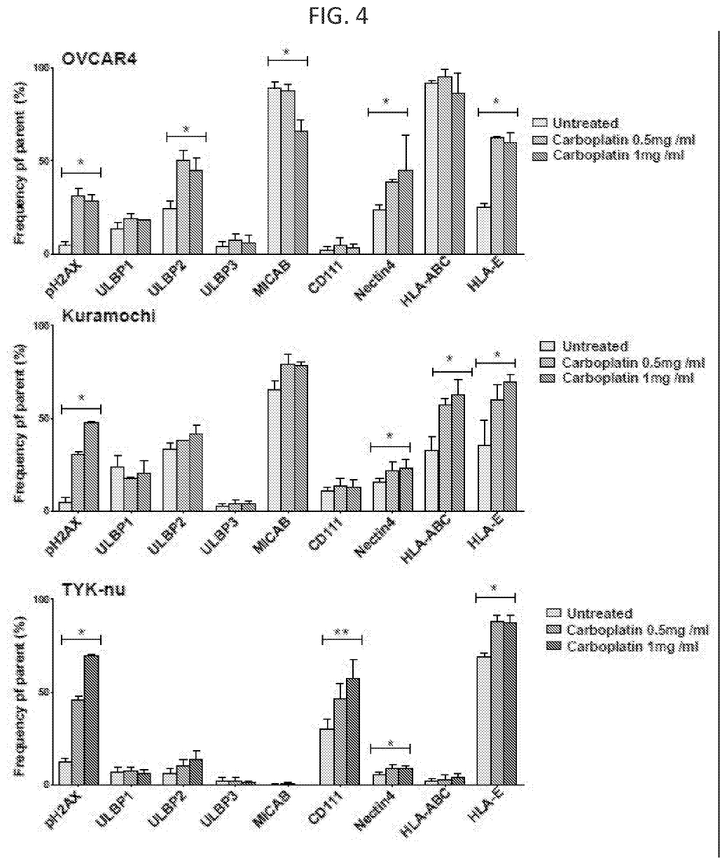

The DNA damage response after carboplatin treatment was confirmed by a recognized increase in pH2AX (FIG. 4). However, of the NK activating receptor ligands measured, only OVCAR4-expressing-ULBP2 cells increased in response to carboplatin (24% to 50%). Evaluation of ligands for inhibitory NK receptors revealed significant increases in frequencies of all three cell lines expressing HLA-E and a significant increase in Kuramochi cells expressing HLA-ABC.

Carboplatin increased the frequency of HGSC cells expressing nectin 4, particularly OVCAR4. Prolonged exposure of cells to carboplatin (at two different doses) increased the frequency of OVCAR4 cells from baseline 24%, to 39%, and 45%. compared to Kuramochi 15%, 22%, and 23% with modest changes in TYK-nu cells (6%, 9%, 9%) (FIG. 4). Overexpression of nectin 4 increases susceptibility to NK cell-mediated cytotoxicity in trophoblasts. Thus, by analogy after carboplatin exposure OVCAR4 showed the greatest susceptibility to NK cell-mediated cytotoxicity with apparently decreasing susceptibility for Kuramochi and TYK-nu cells (FIG. 4). In TYK-nu cells carboplatin mediated a significant increase in CD111, a ligand for the inhibitory CD96 receptor, as well as a role in enhancing signaling through TIGIT. This additional resilience to NK cell cytotoxicity reveals an as yet unrecognized mechanism of carboplatin resistance.

HGSC-NK-92 cell line cocultures to model the HGSC immune tolerant microenvironment. One of the principal findings from our analysis of the HGSC immune cell infiltrate was the positive association between dl-NK cell sub-populations and overall tumor cell abundance. In order to determine whether dl-NK cells, like dNK, are functionally immune-tolerant we measured features of decidual NK cells, using an NK CyTOF antibody panel after in vitro coculture experiments between E, EV and V HGSC cell lines (OVCAR4, Kuramochi and TYK-nu) and the human NK-92 cell line. We chose the NK-92 cell line because of its clinical development for adoptive cellular NK immunotherapy.

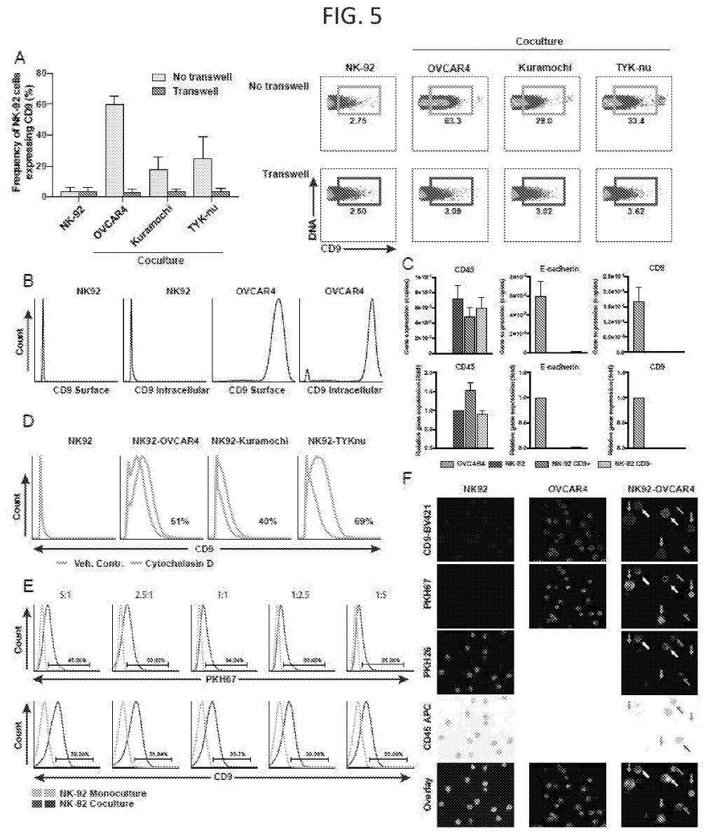

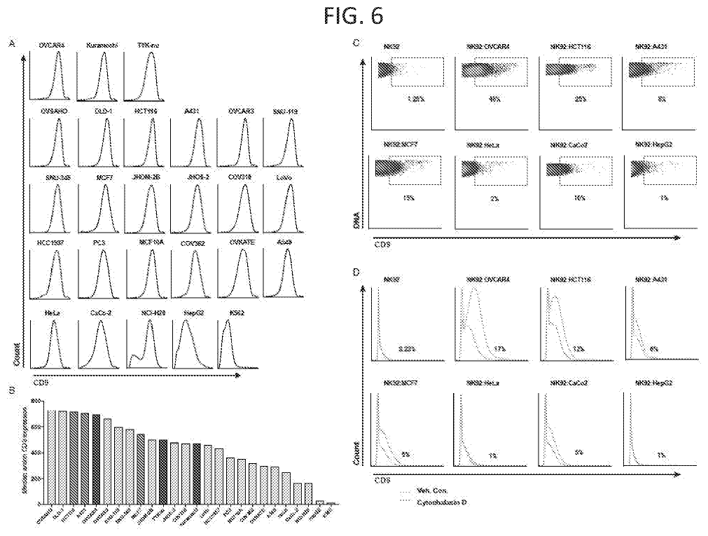

CD9 expression in NK-92 cells after coculture with HGSC cell lines. We first investigated the expression of CD9, a phenotypic hallmark/marker of decidual NK cells. In monoculture, NK-92 cells showed minimal CD9 expression. However, after coculture with HGSC cell lines, up to 60% NK-92 cells expressed CD9. The OVCAR4 cell line mediated the greatest induction of CD9-expressing NK-92 cells that was maximal at 6 h (FIG. 6A) and at 48 h the level was still sustained. When the coculture was performed with a membrane barrier (transwell) between the two cell lines CD9+ expression on NK-92 cells was dramatically reduced (<4%) demonstrating the requirement for physical contact between HGSC tumor and NK-92 cells (FIG. 5A).

One potential explanation for the appearance of surface CD9 expression on NK-92 cells is that they retain intracellular CD9 pools that during coculture with HGSC cells, are induced to traffic to the cell surface. To address this, we stained NK-92 and OVCAR4 cells grown in monoculture with the same CD9 antibody but with different conjugates (Materials and Methods). Sequential cell staining for CD9 (surface, then intracellular) showed that NK-92 cells were devoid of both surface and intracellular CD9. By contrast, OVCAR4 cells expressed robust levels of CD9 in both cellular locations (FIG. 5B and Materials and Methods).

To further confirm that CD9 displayed on NK-92 cells after co-culture was not endogenously produced, CD9+ NK-92 cells and their negative counterparts CD9− NK-92 cells were FACS sorted after coculture with the OVCAR4 cell line and CD9 transcripts measured (FIG. 5C). Transcripts were not detected in either of the FACS sorted NK-92 cells. By contrast, robust levels of CD9 transcripts, consistent with the CD9 protein expression, were seen in the FACS-sorted OVCAR4 line with which they were co-cultured. Control transcripts measured were CD45 (positive for NK92; negative for OVCAR4) and E-cadherin (negative for NK92; positive for OVCAR4) FIG. 5C).

CD9 expression across HGSC primary tumors and cell lines. In order to determine how prevalent CD9 expression was in HGSC we screened 17 primary HGSC tumors and 11 HGSC cell lines to determine both the frequency of CD9-expressing cells and the levels of CD9 expression. For the primary tumor cohort, high frequencies of CD9+ tumor cells were present in all samples with a range of 59-99% across all the 17 samples (FIG. 13). For the HGSC cell lines, we screened the top ranking HGSC cell lines as reported by Domcke et al. and observed high frequencies of CD9-expressing cells. (Note, The HGSC cell lines were screened simultaneously with non-HGSC cell lines shown in FIG. 6A).

Trogocytosis as the mechanism by which NK-92 cells acquire CD9. We hypothesized that HGSC cell lines could be the source of CD9 and transferred to the NK92 cells by a process known as trogocytosis. This involves plasma membrane fragments including anchored proteins being transferred within minutes of cell-cell contact. Therefore, we cocultured NK-92 with OVCAR4 cells for 15, 30, 60, 120 and 360 minutes after which we measured CD9 expression on NK-92 cells by fluorescence-based flow cytometry. We detected CD9 expression on NK-92 cells as early as 15 minutes after coculture, with a steady increase in CD9+ NK-92 cells up to 360 minutes (FIG. 14). These data are consistent with trogocytosis as the mechanism for CD9 acquisition by NK-92 cells. Notably, frequencies of CD9+ NK-92 cells were maintained up to 48 h of coculture and is a result entirely consistent with transfer of membrane-associated proteins in other systems.

To further confirm that the mechanism of CD9 acquisition by NK-92 cells was through trogocytosis we performed an additional series of experiments. Many studies have shown that inhibitors of actin polymerization block trogocytosis but that the effect of these inhibitors vary depending on the cell type. Our pilot experiment tested a series of such trogocytosis inhibitors; concanavalin A, wortmannin, EDTA, nocodazole and cytochalasin D. Optimal results were obtained when NK-92 cells were pre-incubated for 2 h with cytochalasin D after which coculture in the presence of cytochalasin D with OVCAR4, Kuramochi and TYK-nu HGSC tumor cell lines resulted in a 40-69% reduction of CD9+ NK-92 cells (FIG. 5D).

Given that OVCAR4 cells induced the greatest increase in the frequency of CD9+ NK-92 cells (˜60%), subsequent mechanistic experiments were carried out with this HGSC cell line. To confirm that plasma membrane fragments containing CD9 were transferred from OVCAR4 cells, these cells were labelled with PKH67 a green fluorescent lipophilic membrane dye before coculture with NK-92 cells. After 24 h cells were stained with antibodies against CD45 and CD9 and processed for fluorescence-based flow cytometry. At the highest target cell (OVCAR4): effector cell (NK-92) ratios (5:1 and 2.5:1)˜50% of NK-92 cells expressed CD9 and green fluorescent dye was co-detected. As cell ratios decreased to 1:5 so did the capture of OVCAR4 membrane fragments by NK-92 cells (FIG. 5E).

Given that trogocytosis involves the uptake of membrane fragments from donor to recipient cells, we also performed microscopy to visualize the incorporation of CD9+ plasma membrane fragments from OVCAR4 cells into NK-92 cells. OVCAR4 and NK-92 cells were labelled with the lipophilic fluorescent dyes PKH67 (green) and PKH26 (red) respectively. After co-culture for 3 h, cells were stained with antibodies against CD9 and CD45 OVCAR4 cells stained blue (CD9) and green (PKH67) with no signal observed in the red (PKH26) and white (CD45) channels. NK-92 cells were observed in all four channels: red (PKH26), green (PKH67), CD45 (white) and blue (CD9). (FIG. 5F (left-hand columns). These images together with the merged channel image demonstrated that NK-92 have trogocytosed plasma membrane fragments including CD9 from OVCAR4 cells. Trogocytosis in the reverse direction was not detected.

Evaluation of NK-92 trogocyctosis from non-HGSC cell lines. In order to determine whether NK-92 CD9 trogocytosis was a feature of non-HGSC tumor cells, we established the frequency of CD9-expressing cells in 15 non-HGSC as well as 11 HGSC tumor cell lines (FIG. 6A). The frequency and levels of CD9-expressing tumor cells varied across the cell lines and are presented going from highest to lowest CD9 levels (FIGS. 6A and B). When designing our co-culture experiment, we noted that in their trogocytosis study, Daubeuf et al. showed that the expression level of a plasma membrane protein was not a determinant of how well it was transferred to the recipient cell. We therefore chose three non-HGSC tumor cell lines with high CD9 expression (HCT116, A431 and MCF7) and three with lower CD9 expression (HeLa, CaCo-2 and HepG2) for trogocytosis experiments (FIG. 6B). NK-92 trogocytosis from non-HGSC cell lines was highly varied, not correlated to CD9 levels and, aside from the HCT116 colorectal cancer cell line, less pronounced than from the HGSC cell lines (FIGS. 5C and 6C). Cytochalasin D partially inhibited CD9 uptake in NK-92 and was most marked for the HCT116, MCF7 and CaCo2 cell lines (FIG. 6C). The cell line data suggest that in HGSC, NK cells likely acquire CD9 by trogocytosis and although this process may occur in other malignancies it appears to be less pronounced.

Intracellular cytokine production of CD9+ and CD9− NK-92 cells. One of the mechanisms by which dNK cells exert immune tolerance is through poor cytotoxic responses and secretion of a specific set of cytokines. Thus, we hypothesized that trogocytosis and the acquisition of CD9 by NK-92 cells endows them with features of immune tolerance. Therefore, we measured the intracellular expression levels of cytolytic proteins (perforin and granzyme B), anti-tumor cytokines (IL-8, IL-10, TNFα, GM-CSF, IFNγ), the proangiogenic cytokine IL-8, the immunosuppressive cytokine IL-10, and CD107a, a marker for degranulation in both CD9+ and CD9− NK-92 cells after coculture with HGSC cells lines. Specifically, we measured NK-92 cell function before and after coculture in response to phorbol-12-myristate-13-acetate (PMA). Exposing cocultures to PMA circumvents upstream NK receptor signaling and is a convenient method for measuring NK cell function. In a pilot study, we determined that a coculture time of 6 h produced the most robust intracellular cytokine production (ICP). Therefore, NK-92 and HGSC cell lines were cocultured for 6 h in the presence of PMA (4 h exposure) with brefeldin A and monensin added to block secretion (Materials and Methods). For all experiments, we used two positive controls. The first control was PMA-mediated ICP for NK-92 cells growing in monoculture and the second was ICP for NK-92 cells after coculture with K562 cells. Cocultures and controls were subsequently processed for CyTOF analysis using an antibody panel designed to measure degranulation, presence of cytotoxic granules and intracellular cytokine production in NK cells. Importantly, including a CD9 antibody in the CyTOF panel allowed us to gate the CyTOF data files for CD9+ and CD9− NK-92 subpopulations and compare their responses.

CD9+ NK-92 cells have a more immune-suppressive phenotype. For all intracellular proteins assayed, the two metrics we measured were, i) frequency of positive cells and ii) the amount of each protein produced. For a subset of proteins, no differences were observed for NK-92 cells grown in mono- or coculture. Additionally, no differences were observed after coculture for CD9+ and CD9− subpopulations of NK-92 cells. Thus, >85% of NK-92 cells produced equivalently high levels of granzyme B, perforin and MIP1β under all conditions (monocultures, cocultures, presence and absence of PMA). For NK-92 cell degranulation, as determined by CD107a, and production of MIP1a, PMA induced robust responses in mono- and coculture with no differences observed for the latter between CD9+ and CD9− NK-92 subpopulations. VEGF levels were constitutively high with >90% of NK-92 cells producing this angiogenic factor in monoculture and after coculture (FIG. 15).

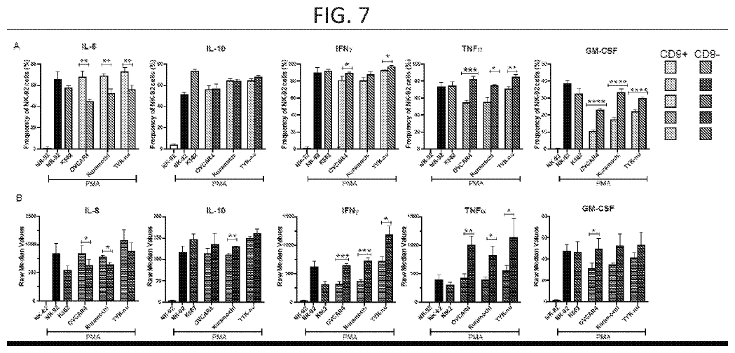

By contrast, after coculture with all three HGSC cell lines and PMA stimulation we observed that a statistically greater proportion of CD9+ NK-92 cells expressed IL-8 compared to CD9− NK-92 cells (FIG. 7A). IL-8 is a proangiogenic factor produced by dNK cells with a role in vascularizing the placenta and in the context of malignancy has a role in promoting the tumor angiogenic system. Moreover, after coculture and PMA stimulation, the proportion of NK-92 cells that expressed TNFα, GM-CSF and IFNγ was significantly lower in CD9+ NK-92 cells. For the immunosuppressive cytokine IL-10, ˜60% of CD9+ and CD9− NK-92 cells produced this cytokine. A further consideration as to how CD9+ NK-92 cells could modulate the tumor microenvironment is by regulating the amount of cytokine produced (FIG. 7B). Consistent with their immune tolerant function, CD9+ NK-92 cells produced statistically reduced amounts of anti-tumor cytokines TNFα, GM-CSF and IFNγ compared to CD9− cells in the same cocultures.

The intracellular cytokine data strongly suggest that CD9-expressing NK-92 cells have a more immunosuppressive function through modulating cytokine production both in terms of frequency of cells and amounts produced.

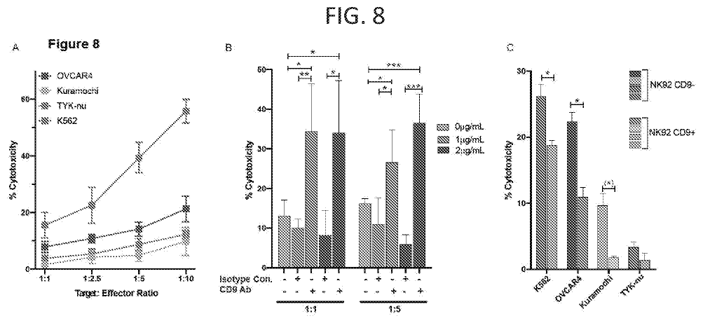

NK-92 cell cytotoxicity attenuated by HGSC cell lines. One of the hallmarks of dNK cells is their low cytotoxicity that is consistent with conferring immune tolerance to the fetus. To determine whether this hallmark was evident when NK-92 cells were cocultured with HGSC cell lines we performed in vitro cytotoxicity assays. NK-92 cells were cocultured with OVCAR4, Kuramochi and TYK-nu cell lines for 4 h after which calcein release was measured (FIG. 8A, Materials and Methods). Compared to the K562 cell line, the most sensitive target cell for human NK cells, NK-92 cytotoxicity was significantly reduced toward all three HGSC cell lines. Furthermore, the magnitude of attenuation trended with stage of tumor progression such that OVCAR4, cells were more and TYK-nu cells less susceptible to NK cell killing. This result is consistent with the NK receptor ligand profile of V cells that is more suppressive toward NK cell function than either E or EV HGSC tumor cell types (FIGS. 2 and 3).

Preincubation with a blocking CD9 antibody restores NK-92 cell cytotoxicity. In order to determine whether the reduced cytotoxicity observed toward HGSC cell lines could be attributed either to CD9 or to other plasma membrane proteins simultaneously transferred to NK-92 cells by trogocytosis, we performed the cytotoxicity assay in the presence of a CD9 blocking antibody (FIG. 8B). The data showed that the CD9 blocking antibody significantly increased NK-92-mediated cytotoxicity toward OVCAR4 cells. These data make a strong case for CD9 having a prominent role in HGSC immunosuppression.

FACS-sorted CD9 NK-92 cells have reduced cytotoxicity. In order to directly compare the cytotoxicity between CD9+ and CD9− NK-92 cells, we FACS-sorted CD9+ NK-92 cells after coculture with OVCAR4. The calcein release cytotoxicity assay revealed statistically significant attenuation of killing activity by CD9+ NK92 cells compared to NK-92 cells grown in monoculture (low background CD9 protein and mRNA) (FIG. 8B). For K562 cells the attenuation was 33%, for OVCAR4 49%. and for Kuramochi 75% Since cytotoxicity toward the TYK-nu cell line was very low at the start further reduction in killing was minimal and not statistically significant. These data demonstrate that the presence of CD9 on NK-92 cells endows them with a more immune-tolerant phenotype.

Reactivating the immune system with immune checkpoint inhibitors to overcome host immune tolerance is a break-through therapeutic approach in the field of oncology. However, data from several clinical trials for women with HGSC receiving immune checkpoint inhibitors have been disappointing. One explanation for these data could be the existence of additional cell types that over-ride any reversal of T-cell mediated immune suppression. Consistent with this, our CyTOF data analysis of newly diagnosed chemo-naïve HGSC tumors revealed previously unidentified dl-NK cell subpopulations that were positively correlated with the overall abundance of tumor and EV cells (FIG. 1 and FIG. 10A). The presence of dl-NK cells have been reported in colorectal and lung tumors, but to our knowledge this is the first report of this immune cell type in HGSC.

Decidual NK cells comprise 70% of the total lymphocyte population during the first trimester of pregnancy and are phenotypically and functionally distinct from peripheral NK cells. They produce a wide range of secretory proteins that are critical for decidualization, formation and vascularization of the placenta and creation of a privileged immune tolerant maternal-fetal compartment. Furthermore, although dNK cells are poorly cytotoxic they contain cytotoxic granules which can be transiently activated to provide immunity to infection during pregnancy.

This study was based on the hypothesis that dl-NK cells have coopted decidual properties to create an immune tolerant and pro-angiogenic tumor microenvironment and that HGSC tumor cells manipulate dl-NK properties toward this end. Our approach capitalized on CyTOF to perform the first reported multiparameter single cell analysis of NK receptor ligand expression on newly diagnosed chemo-naïve late-stage HGSC tumors and additionally, with in vitro studies, determine how HGSC tumor cells modulate NK cell function. Overall, the data in this study reveal previously unrecognized mechanisms of immune suppression mediated by HGSC tumor cells. This includes combinatorial expression of ligands that bind to NK receptors as well as the ability to transfer of CD9 by trogocytosis onto NK and likely other immune cell types.

Data analysis revealed that E and EV cells expressed more NK activating receptor ligands than V cells consistent with previous reports that cells undergoing epithelial to metastatic transition lose NKG2D ligands. An exception to these findings were high levels of the NKG2D ligand, ULBP4, a functional outlier within the ULBP family, that may promote immune escape rather than cytotoxicity. Consistent with this notion, recent studies showed that proteolytic shedding and/or the generation of exosomes constitutively expressing ULBP4 down regulated activating NKG2D receptors as alternative mechanisms for immune escape.

Boolean and Simpson's analysis revealed that the E and EV compartments had greater combinatorial diversity (103 and 101 combinations respectively) for NK receptor ligands than the V tumor compartment (67) (FIG. 3). These data suggest that the greater number of ligand combinations in the earlier stages of tumor development could increase the likelihood of tumor-immune cell escape and eventual transition into poor-prognosis V cells. Having attained a V phenotype, these tumor cells may then switch to an alternative more stringent immune escape mechanism. (FIG. 2D and FIG. 3). The consequence of this is that the V cell HGSC microenvironment is likely to be hostile to NK cell immunotherapy.

Numerous studies documented upregulation of NK receptor activating ligands in response to exposure to genotoxic agents, epigenetic modifiers and radiation. This approach has the potential, to make HGSC (especially the metastatic V compartment) more amenable to NK immunotherapy. We exposed the E, EV and V HGSC, cell lines to carboplatin, a chemotherapeutic agent used in first line HGSC treatment regimens (FIG. 4). In all three cell lines, carboplatin increased the proportion of HGSC tumor cells that expressed NK inhibitory receptor ligands. Additionally, the increased frequencies of tumor cells expressing nectin 4 and CD111 may not be restricted to their ability to activate NK cells, since both nectin 4 and CD111 have additional roles in adhesion, cell movement and stem cell biology. Although, nectin 4 expression could enhance tumor cell susceptibility to NK cell cytotoxicity, it has been proposed to have a role in HGSC metastasis and chemotherapeutic resistance. The potential of carboplatin to create a more immune inhibitory microenvironment is an unrevealed mechanism for platinum resistance in HGSC, which remains a major challenge to improving outcomes for patients with HGSC.

In order to gain mechanistic insight into whether the decidual-like NK cell phenotype was also accompanied by decidual NK cell functional attributes we designed coculture experiments between the three HGSC cell lines that modelled the E, E-V, V tumor compartments with the NK-92 cell line. The NK-92 cell line expresses CD56, lacks expression of inhibitory KIR receptors and is molecularly well-characterized. NK-92 cells have been genetically engineered to express chimeric antigen receptors, activating NK receptors such as NKG2D, and in several early phase clinical trials have satisfied safety criteria.

In coculture, we demonstrated that E, EV and V HGSC cell lines induced expression of the tetraspanin CD9 by trogocytosis with the greatest induction consistently observed for OVCAR4 cells (FIG. 5A-F). The high levels of CD9 expression we observed on HGSC cells lines and absence of its expression on NK-92 cells were consistent with this mechanism. Trogocytosis, a process in which fragments of the plasma membrane containing anchored proteins are transferred from one cell to the next, has been observed in T cells, B cells basophils and NK cells. In those studies, trogocytosis occurred between different immune cell phenotypes and endowed the recipient immune cell phenotype with de novo functional properties and opportunities to interact with other previously inaccessible immune cell types. Trogocytosis has also been observed to occur between immune and non-immune cell types. For example, in two separate studies, in vitro coculture experiments between dNK cells with extravillous trophoblasts, and peripheral NK cells with melanoma cells (respectively) resulted in the transfer of HLA-G onto the NK cells. In so doing NK cell immune tolerance in both systems was enhanced. Additionally, a recent report described trogocytosis as a potential mechanism of resistance to chimeric antigen T (CAR T) cell therapy in patients. Using a murine leukemic model, CAR T cells acquired CD19, the target antigen from tumor cells by trogocytosis, a result consistent with the reduced CD19 levels seen in patients' tumors. These studies provide ever-increasing evidence for trogocytosis playing a key role in immune tolerance and more recently therapeutic resistance.

The high levels of CD9 expression by HGSC cell lines and primary tumors provide a source of CD9 for intra-tumoral NK cells (FIG. 7 and FIG. 13). Furthermore, other tumor-infiltrating immune cell types may also acquire CD9 by trogocytosis exemplified by the presence of two T cell clusters with high levels of CD9 expression (FIG. 10A). In order to determine whether the high CD9 expression levels were a property of HGSC, we screened a series of non-HGSC tumor cell lines for CD9 expression (FIG. 7A, B). CD9 was ubiquitously expressed but at different levels across the non-HGSC cell lines. Furthermore, when compared to the OVCAR4 cell line, trogocytosis was observed but often greatly diminished (FIG. 7C). Based on these data, trogocytosis is likely to occur in most tumors, including dl-NK cells reported in colorectal and lung cancer.

While the exclusivity of CD9 expression on the decidual NK subpopulation is well established, its functional role in these cells is unclear. In this study, data analysis from coculture experiments showed that gain of CD9 expression by NK-92 cells through trogocytosis coincided with significant decreases in immune modulatory cytokines (IFNγ, TNFα and GM-CSF), increases in the proangiogenic cytokine IL-8 and suppressed cytotoxicity (FIGS. 7 and 8). Critically, a CD9 blocking antibody significantly increased NK-92 cytotoxicity providing strong evidence that CD9 confers NK-92 cells with hyporesponsive properties (FIG. 9B). Furthermore, hyporesponsive NK cells isolated from ovarian ascites fluid were shown to have decreased levels of the activating receptor DNAM1. Together these findings highlight multiple independent mechanisms tumors use to suppress NK cell function.

CD9 shows ubiquitous distribution and is involved in multiple cellular functions such as proliferation, motility and adhesion with major roles in formation of the immune cell synapse. It is thus likely that CD9 may have multiple roles in regulating the HGSC tumor immune microenvironment. It has been shown to directly associate with ADAM17 protease and inhibit its cleavage activity toward surface protein ectodomains. Thus, transfer of CD9 from HGSC tumor cells onto NK-92 cells could reactivate ADAM17 toward NK activating receptor ligand substrates thereby facilitating yet another mechanism for immune escape.

The significance of our results underscores the critical need to evaluate NK receptor ligand expression within the tumor microenvironment(s) for HGSC patients eligible to receive NK cell immunotherapy. This is especially important for patients in a relapse setting, as currently these patients would be those most likely to receive this therapy. In order to maximize NK cell cytotoxicity (both intra-tumoral and adoptively administered) there must necessarily be a favorable balance in expression levels of NK receptor activating ligands.

The abundant expression of CD9 in primary HGSC tumor cells (FIG. 14) presents the distinct possibility that NK-92 or other adoptively transferred NK cells could acquire CD9 by trogocytosis and transition to a more immunosuppressive phenotype thereby negating the intention of the therapy. Thus, a peripheral blood test to monitor gain of CD9 expression by adoptively transferred NK cells is of interest. Additionally, a blocking CD9 antibody could be administered before NK immunotherapy. Converting the mechanistic insight revealed by this study into biomarkers is relevant to all forms of NK immunotherapy. Such biomarkers could not only guide the selection of those HGSC patients most likely to respond to NK immunotherapy, but can be used to monitor the durability of patient responsiveness. This study undoubtedly has relevance for other malignancies for which NK immunotherapy is an option.

### Materials and Methods

Patient Samples. Deidentified newly diagnosed chemo naïve HGSC tumors prepared as single cell suspensions for CyTOF analysis collected over a two-year period were purchased from Indivumed (Hamburg, Germany). Tumor samples were collected in compliance with the Helsinki declaration and all patients provided written informed consent. The use of human tissue was approved and complied with data protection for patient confidentiality. Institutional review board approval was obtained at the Physicians Association in Hamburg, Germany.

Genomic sequencing and analysis for TP53 and BRCA1/2. DNA was extracted and enriched through multiplex PCR (QIAGEN QIAmp DNA Mini-Kit and QIAGEN GeneRead DNaseq Targeted Ovarian V2 Panel, respectively). The TrueSeq protocol was used to make an indexed illumina sequencing library from the pooled sample amplicons. The subsequent protocols for sequencing were described previously. The pathogenic variants were noted.

Cancer cell lines from HGSC and non-HGSC malignancies. Cell lines were authenticated by short tandem repeat (STR) profiling performed by the Stanford Functional Genomics Facility. Ovarian cancer cell lines (OVCAR4, Kuramochi, and TYK-nu), NK-92 and other non-HGSC cell lines used for CD9 screen were grown according to the recommended conditions from their respective vendors.

Antibodies for CyTOF. Antibodies were either purchased pre-conjugated or conjugated in-house as previously reported In brief, for in-house conjugations, antibodies in carrier-free PBS were conjugated to metal-chelated polymers (MaxPAR antibody conjugation kit, Fluidigm) according to the manufacturer's protocol or to bismuth with our protocol(101). Metal-labeled antibodies were diluted to 0.2-0.4 mg/mL in antibody stabilization solution (CANDOR Biosciences) and stored at 4° C. Each antibody was titrated using cell lines and primary human samples as positive and negative controls. Antibody concentrations used in experiments were based on an optimal signal-to-noise ratio. Three CyTOF antibody panels were used in this study to characterize: i) tumor T and NK cells ii) Tumor NK receptor ligand expression and iii) NK cell receptor and intracellular cytokine expression.

Antibodies for Fluorescence-Based Flow Cytometry. Antibodies were purchased for detection of CD9 and CD45 from Becton Dickinson (CD9 BV421, CD9 PE) and Biolegend (CD45 APC). The same antibody clones were used for CyTOF. Near-IR fixable LIVE/DEAD stain from Thermofisher was used to distinguish dead cells.

Sample Processing and Antibody Staining for CyTOF. Frozen, fixed single-cell suspensions of HGSC tumors or cell lines were thawed at room temperature. For each sample, 1×10⁶ cells were aliquoted into cluster tubes in 96 well plates and subjected to pre-permeabilization palladium barcoding. After barcoding, pooled cells were pelleted and incubated for 10 min at room temperature with FcX block (Biolegend) to block non-specific antibody binding. Cells were then incubated with antibodies against surface markers for 45 min at room temperature. Cells were permeabilized at 4° C. with methanol or 1× Permeabilization Buffer (eBioscience) (only for the CyTOF antibody panel designed to characterize intracellular cytokines, on ice for 10 min. Cells were subsequently stained with antibodies against intracellular markers for 1 h at room temperature, washed, and incubated with the ^{191/193}Ir DNA intercalator (Fluidigm) at 4° C. overnight. Cells were washed and resuspended in a solution of normalization beads before introduction into the CyTOF 2.

Determination of intracellular pools of CD9 by CyTOF. NK-92 and OVCAR4 cells were stained with cisplatin (Sigma Aldrich), fixed with 1.6% paraformaldehyde (ThermoFisher), washed and incubated with FcX block (Biolegend) for 10 min at room temperature. Cells were then incubated with CD9-PE (Becton Dickinson) for 45 min at room temperature. Cells were washed and stained with anti-PE-165Ho (Fluidigm) for 30 min at room temperature. Following secondary antibody staining, cells were permeabilized with 1× Permeabilization Buffer (eBioscience) on ice for 10 min. Cells were subsequently stained with CD9-156Gd (Fluidigm) for 1 h at room temperature to detect intracellular expression levels of CD9. Cells were processed before introduction into the CyTOF 2 as described above.

In vitro cocultures to determine intracellular cytokine production of NK-92 cells. The HGSC cell lines, OVCAR4, Kuramochi and TYK-nu cells, were each cocultured with the NK-92 cell line, at an effector:target ratio of 1:1 for 6 h unless otherwise indicated, at 37° C. in a humidified cell culture incubator. HGSC cells (100,000 cells/well) were seeded in U-bottom 96-well plates (Corning, Costar) with NK-92 cells (100,000). During the last 4 h of coculture, PMA/lonomycin cell stimulation cocktail (500×) (eBioscience) was added to induce intracellular cytokine production. The protein transport inhibitors, Brefeldin A and Monensin (eBioscience, ThermoFisher), were used at a final concentration of 3 μg/ml and 2 μM, respectively. There were two positive controls; NK-92 cells grown in monoculture −/+PMA and coculture between the K562 cell line (HLA-null erythroleukemic)(100) with NK-92 cells. CD107a-151Eu antibody (Fluidigm) (1 μl) was added to each well as marker for degranulation. All experiments were performed with biological and technical triplicates with details described for specific assays.

Transwell assay. OVCAR4, Kuramochi and TYK-nu cells were cocultured with NK-92 in a 96-well dual-chamber transwell plate with 3 μm micropores (Corning, Costar). HGSC cells (100,000/well) were placed into the lower chamber, and NK-92 cells (100,000/well) were placed into the upper chamber. The cells were cultured at 37° C. for 6 h in a humidified cell culture incubator. The assay was performed with biological and technical triplicates.

Trogocytosis. OVCAR4 were labeled with PKH67 (Sigma Aldrich) prior to coculture with NK-92. In brief, OVCAR4 were washed with serum free media and resuspended in diluent C. A 2× working solution of PKH67 was prepared immediately prior to use. Cells were mixed with PKH67 working solution for a final concentration of 5×10⁶ cells/mL in 20 μM PKH67 and incubated for 5 min at room temperature. The labeling was quenched with an equal volume of fetal bovine serum, incubated for 1 min, and washed three times with 10 mL of complete media. Cells were seeded in U-bottom 96-well plates (Corning, Costar), and cocultured with NK-92 cells at target:effector ratios 5:1, 2.5:1, 1:1, 2.5:1 and 5:1 for 24 h at 37° C. Cells were then stained with CD45 and CD9 antibodies and processed for flow cytometry.

Reverse transcriptase quantitative PCR to measure CD9 transcript levels. Total mRNA was isolated with a Qiagen MicroRNA isolation kit from OVCAR4 and NK-92 cells grown in monoculture and FACS-sorted CD9+ and CD9− NK-92 cells after coculture with OVCAR4 cells. cDNA was generated using the High Capacity cDNA Reverse Transcription Kit from Applied Biosystems according to manufacturer's protocol. Real-time PCR for E-cadherin (CDH1) Hs00170423_m1, CD45 (PTPRC) Hs00894716_m1, and CD9 Hs01124022 was performed with a Taqman gene expression kit and run on the ABI 7900HT instrument.

Microscopy to image trogocytosis. OVCAR4 and NK92 cells were labeled with the membrane dyes PKH67 and PKH26(Sigma Aldrich), respectively, prior to coculture. Cells were washed with serum free media and resuspended in Diluent C. A 2× working solution of each membrane dyes was prepared immediately prior to use. Cells (5×10⁶ cells/mL) were mixed with their respective working solution of dye for a final concentration of 20 mM. After a 5 min incubation at room temperature the labeling was quenched with an equal volume of fetal bovine serum, incubated for 1 min, and washed three times with 10 mL of complete media. Cells were seeded in U-bottom 96-well plates (Corning, Costar), and cocultured with NK-92 cells at an effector:target ratio 1:1 for 3 h at 37° C. Cells were then fixed with a final concentration of 1.6% paraformaldehyde, stained with CD45 and CD9 antibodies and seeded on microscope slides for imaging on a Keyence BZ-X800 microscope.

Trogocytosis inhibition. NK-92 cells were pre-treated with cytochalasin D (10 μM in complete media) for 2 h. They were then co-cultured with cancer cell lines as indicated in a ratio of 1:1 in the continuous presence of cytochalasin D for a further 2 h after which cells were stained with antibodies against CD9, CD45 and processed prior to CyTOF analysis as described above.

Calcein-AM release cytotoxicity assay. The calcein release assay was performed according to published conditions. OVCAR4, Kuramochi, TYK-nu and K562 (control) target cells were washed in PBS and resuspended in calcein-acetoxymethyl (calcein-AM; ThermoFisher) staining solution (2.5 μM in PBS) at a cell density of 1×10⁶/mL and incubated for 30 min at 37° C. Target cells were seeded in U-bottom 96-well plates (Corning, Costar), and cocultured with NK-92 cells at increasing effector:target ratios 1:1, 2.5:1, 5:1 and 10:1 for 4 h at 37° C. For the cytotoxicity assay with CD9 blocking antibody, after both cells lines were plated either control mouse IgG1k, (Abcam C #ab170190, clone: 15-6E10A7) or purified mouse monoclonal CD9 antibody (Abcam C #2215, clone: MEM-61) were added to the coculture for the duration of the incubation. Cells were then spun down and 100 μl of supernatant were transferred to a black-walled 96 well plate (Corning, Costar). Calcein release was measured from the fluorescent signal using 485 nm excitation wavelength and 530 nm emission wavelength (Ex/Em Calcein: 494/517 with a Tecan Infinite M1000 fluorescent plate reader. Control wells contained HGSC target cells alone (spontaneous lysis) or with 2% Tween-20 (maximum lysis). Specific killing was calculated using the equation: specific killing=(lysis of coculture−spontaneous lysis)/(maximum lysis−spontaneous lysis)×100%. The assay was performed with biological and technical quadruplets.

Data analysis tools. All data and statistical analysis were implemented with Microsoft Excel, Matlab, R and Graph Pad Prism 8. CyTOF datasets were evaluated with software available from Cytobank and CellEngine (Primity Bio). Boolean analysis to determine NK receptor ligand combinations were determined using FlowJo V10.

Initial Assessment of data quality. Initial data quality was assessed by determining dead and apoptotic cells which were excluded from further analysis. Viable cells, defined as cisplatin negative and cleaved PARP negative were used for experiments. For experiments with newly diagnosed HGSC tumors, tumor cells were gated as CD45−/CD31−/FAP− and immune cells were gated as CD45+ CD66− as described previously.

Clustering of tumor and T and NK cell immune infiltrate. Manually gated CD45−/CD31−/FAP− cells from all 12 newly diagnosed HGSC tumors were pooled for clustering with X-shift, a density-based clustering algorithm, using the Vortex clustering environment.

Correlation network analysis. Spearman pairwise correlation coefficients (rₛ) were calculated from the CyTOF data of the tumor cells and the analysis of the same tumors with a CyTOF antibody panel designed to interrogate T and NK cells. Correlations were computed between: i) cell frequencies for 56 tumor cell clusters, ii) frequency of 52 T and NK cell clusters, iii) total tumor cell abundance, iv) total EV cell abundance, v) total E-cadherin cell abundance and vi) other features previously described. A hierarchically ordered heat-map was generated in R.

Force directed layout visualization. Force directed layouts were generated from a composite of all 12 HGSC tumors. After merging all single cell data files, cells were clustered. 10,000 single cells were computationally sampled from each of the 56 tumor cell clusters. Each cell was connected on a 10-nearest-neighbor graph. This graph was subjected to a force-directed layout (FDL) that placed groups of phenotypically related cells adjacent to one another(22). Repeat samplings generated comparable results. Layouts were colored for expression of E-cadherin, vimentin, NK receptor ligands or ADAM10 and ADAM17.

Combinatorial expression for NK receptor ligands in E, EV and V tumor compartments. Viable tumor cells were manually gated from newly diagnosed HGSC tumors as negative for cisplatin and cPARP. As previously described, the tumor population was gated as CD45−/FAP−/CD31−. The E, EV and V tumor compartments were subsequently gated from this viable tumor cell population. Frequencies of tumor cell subpopulations defined by their combinatorial expression patterns of the twelve NK receptor ligands and two ADAM proteases were determined with MATLAB. For this analysis, the frequency of tumor cells expressing each of these proteins was determined in each compartment on a per sample basis. Combinations used in the analysis were based on a threshold frequency of >1% for cells in any compartment in any sample.

Simpson's index of diversity. This analysis was performed in Excel. The Simpson's index of diversity, D was calculated with the formula

$$D = 1 - ( \frac{\sum n ( n - 1 )}{N ( N - 1 )} )$$

N is the total number of tumor cell subpopulations with a specific NK receptor ligand combination (163) and n is the number of times a subpopulation is present in the E, EV and V compartments of each of the 12 tumors (FIG. 3C).

### REFERENCES

1. N. Singh, W. G. McCluggage, C. B. Gilks, High-grade serous carcinoma of tubo-ovarian origin: recent developments. Histopathology 71, 339-356 (2017).
2. R. C. Bast, Jr. et al., Critical Questions in Ovarian Cancer Research and Treatment: Report of an American Association for Cancer Research Special Conference. (2019).
3. D. D. Bowtell et al., Rethinking ovarian cancer II: reducing mortality from high-grade serous ovarian cancer. Nat Rev Cancer 15, 668-679 (2015).
4. U. A. Matulonis et al., Ovarian cancer. Nat Rev Dis Primers 2, 16061 (2016).
5. C. J. Lord, A. Ashworth, PARP inhibitors: Synthetic lethality in the clinic. Science 355, 1152-1158 (2017).
6. A. Ashworth, C. J. Lord, Synthetic lethal therapies for cancer: what's next after PARP inhibitors? Nat Rev Clin Oncol 15, 564-576 (2018).
7. G. M. Rodriguez, K. J. C. Galpin, The Tumor Microenvironment of Epithelial Ovarian Cancer and Its Influence on Response to Immunotherapy. 10, (2018).
8. L. Lee, U. Matulonis, Immunotherapy and radiation combinatorial trials in gynecologic cancer: A potential synergy? Gynecol Oncol, (2019).
9. L. E. Kandalaft, K. Odunsi, G. Coukos, Immunotherapy in Ovarian Cancer Are We There Yet? J Clin Oncol 37, 2460-2471 (2019).
10. M. T. Orr, L. L. Lanier, Natural killer cell education and tolerance. Cell 142, 847-856 (2010).
11. E. Vivier et al., Innate or adaptive immunity? The example of natural killer cells. Science 331, 44-49 (2011).
12. A. J. Wilk, C. A. Blish, Diversification of human NK cells: Lessons from deep profiling. J Leukoc Biol 103, 629-641 (2018).
13. M. G. Morvan, L. L. Lanier, NK cells and cancer you can teach innate cells new tricks. Nat Rev Cancer 16, 7-19 (2016).
14. L. Chiossone, P. Y. Dumas, M. Vienne, E. Vivier, Natural killer cells and other innate lymphoid cells in cancer. Nat Rev Immunol 18, 671-688 (2018).
15. V. D. Gonzalez et al., Commonly Occurring Cell Subsets in High-Grade Serous Ovarian Tumors Identified by Single-Cell Mass Cytometry. Cell reports 22, 1875-1888 (2018).
16. M. Daher, K. Rezvani, Next generation natural killer cells for cancer immunotherapy: the promise of genetic engineering. Curr Opin Immunol 51, 146-153 (2018).
17. Y. Li, D. L. Hermanson, B. S. Moriarity, D. S. Kaufman, Human iPSC-Derived Natural Killer Cells Engineered with Chimeric Antigen Receptors Enhance Anti-tumor Activity. Cell Stem Cell 23, 181-192.e185 (2018).
18. L. D. Uppendahl, C. M. Dahl, J. S. Miller, M. Felices, M. A. Geller, Natural Killer Cell-Based Immunotherapy in Gynecologic Malignancy: A Review. Frontiers in immunology 8, 1825 (2017).
19. S. Lorenzo-Herrero et al., NK Cell-Based Immunotherapy in Cancer Metastasis. Cancers 11, (2018).
20. K. Rezvani, Adoptive cell therapy using engineered natural killer cells. Bone Marrow Transplant 54, 785-788 (2019).
21. K. Rezvani, R. Rouce, E. Liu, E. Shpall, Engineering Natural Killer Cells for Cancer Immunotherapy. Mol Ther 25, 1769-1781 (2017).
22. N. Samusik, Z. Good, M. H. Spitzer, K. L. Davis, G. P. Nolan, Automated mapping of phenotype space with single-cell data. Nat Methods, (2016).
23. Y. Zhang, R. A. Weinberg, Epithelial-to-mesenchymal transition in cancer complexity and opportunities. Frontiers of medicine 12, 361-373 (2018).
24. M. H. Spitzer et al., Systemic Immunity Is Required for Effective Cancer Immunotherapy. Cell, (2017).
25. T. Ideker, N. J. Krogan, Differential network biology. Molecular systems biology 8, 565 (2012).
26. A. N. Hotson et al., Coordinate actions of innate immune responses oppose those of the adaptive immune system during Salmonella infection of mice. Science signaling 9, ra4 (2016).
27. A. Horowitz et al., Genetic and environmental determinants of human NK cell diversity revealed by mass cytometry. Science translational medicine 5, 208ra145 (2013).
28. L. A. Koopman et al., Human decidual natural killer cells are a unique NK cell subset with immunomodulatory potential. J Exp Med 198, 1201-1212 (2003).
29. C. H. Kim, E. C. Butcher, B. Johnston, Distinct subsets of human Valpha24-invariant NKT cells: cytokine responses and chemokine receptor expression. Trends in immunology 23, 516-519 (2002).
30. R. Reyes, B. Cardenes, Y. Machado-Pineda, C. Cabanas, Tetraspanin CD9: A Key Regulator of Cell Adhesion in the Immune System. Frontiers in immunology 9, 863 (2018).
31. A. Koreck et al., CD3+CD56+ NK T cells are significantly decreased in the peripheral blood of patients with psoriasis. Clin Exp Immunol 127, 176-182 (2002).
32. H. B. Bemstein et al., CD4 expression on activated NK cells: ligation of CD4 induces cytokine expression and cell migration. J Immunol 177, 3669-3676 (2006).
33. A. Lopez-Soto, S. Gonzalez, M. J. Smyth, L. Galluzzi, Control of Metastasis by NK Cells. Cancer Cell 32, 135-154 (2017).
34. J. Hanna, O. Mandelboim, When killers become helpers. Trends in immunology 28, 201-206 (2007).
35. M. A. Cooper, T. A. Fehniger, M. A. Caligiuri, The biology of human natural killer-cell subsets. Trends in immunology 22, 633-640 (2001).
36. J. Hanna et al., Decidual NK cells regulate key developmental processes at the human fetal-maternal interface. Nature medicine 12, 1065-1074 (2006).
37. N. Jabrane-Ferrat, Features of Human Decidual NK Cells in Healthy Pregnancy and During Viral Infection. Frontiers in immunology 10, 1397 (2019).
38. P. Dhar, J. D. Wu, NKG2D and its ligands in cancer. Curr Opin Immunol 51, 55-61 (2018).
39. D. H. Raulet, S. Gasser, B. G. Gowen, W. Deng, H. Jung, Regulation of ligands for the NKG2D activating receptor. Annu Rev Immunol 31, 413-441 (2013).
40. P. Boutet et al., Cutting edge: the metalloproteinase ADAM17/TNF-alpha-converting enzyme regulates proteolytic shedding of the MHC class I-related chain B protein. J Immunol 182, 49-53 (2009).
41. L. Ferrari de Andrade et al., Antibody-mediated inhibition of MICA and MICB shedding promotes NK cell-driven tumor immunity. Science 359, 1537-1542 (2018).
42. L. Martinet, M. J. Smyth, Balancing natural killer cell activation through paired receptors. Nat Rev Immunol 15, 243-254 (2015).
43. S. Fabre-Lafay et al., Nectin-4 is a new histological and serological tumor associated marker for breast cancer. BMC Cancer 7, 73 (2007).
44. B. Sanchez-Correa et al., DNAM-1 and the TIGIT/PVRIG/TACTILE Axis: Novel Immune Checkpoints for Natural Killer Cell-Based Cancer Immunotherapy. Cancers 11, (2019).
45. E. E. Wroblewski, P. Parham, L. A. Guethlein, Two to Tango: Co-evolution of Hominid Natural Killer Cell Receptors and MHC. Frontiers in immunology 10, 177 (2019).
46. T. Kamiya, S. V. Seow, D. Wong, M. Robinson, D. Campana, Blocking expression of inhibitory receptor NKG2A overcomes tumor resistance to NK cells. J Clin Invest 130, 2094-2106 (2019).
47. T. Zoller, M. Wittenbrink, M. Hoffmeister, A. Steinle, Cutting an NKG2D Ligand Short Cellular Processing of the Peculiar Human NKG2D Ligand ULBP4. Frontiers in immunology 9, 620 (2018).
48. S. C. Wei, C. R. Duffy, J. P. Allison, Fundamental Mechanisms of Immune Checkpoint Blockade Therapy. Cancer discovery 8, 1069-1086 (2018).
49. C. A. Brennick, M. M. George, W. L. Corwin, P. K. Srivastava, H. Ebrahimi-Nik, Neoepitopes as cancer immunotherapy targets: key challenges and opportunities. Immunotherapy 9, 361-371 (2017).
50. J. M. Vyas, A. G. Van der Veen, H. L. Ploegh, The known unknowns of antigen processing and presentation. Nat Rev Immunol 8, 607-618 (2008).
51. S. Domcke, R. Sinha, D. A. Levine, C. Sander, N. Schultz, Evaluating cell lines as tumour models by comparison of genomic profiles. Nature communications 4, 2126 (2013).
52. C. Cerboni et al., The DNA Damage Response: A Common Pathway in the Regulation of NKG2D and DNAM-1 Ligand Expression in Normal, Infected, and Cancer Cells. Frontiers in immunology 4, 508 (2014).
53. S. Gasser, S. Orsulic, E. J. Brown, D. H. Raulet, The DNA damage pathway regulates innate immune system ligands of the NKG2D receptor. Nature 436, 1186-1190 (2005).
54. L. Krenning, J. van den Berg, R. H. Medema, Life or Death after a Break: What Determines the Choice? Mol Cell, (2019).
55. M. Ito et al., Potential role for nectin-4 in the pathogenesis of pre-eclampsia: a molecular genetic study. BMC medical genetics 19, 166 (2018).
56. E. Joly, D. Hudrisier, What is trogocytosis and what is its purpose? Nat Immunol 4, 815 (2003).
57. A. Dance, Core Concept: Cells nibble one another via the under-appreciated process of trogocytosis. Proc Natl Acad Sci USA 116, 17608-17610 (2019).
58. J. Reed, S. A. Wetzel, Trogocytosis-Mediated Intracellular Signaling in CD4(+) T Cells Drives TH2-Associated Effector Cytokine Production and Differentiation. J Immunol 202, 2873-2887 (2019).
59. A. Aucher, E. Magdeleine, E. Joly, D. Hudrisier, Capture of plasma membrane fragments from target cells by trogocytosis requires signaling in T cells but not in B cells. Blood 111, 5621-5628 (2008).
60. R. Gary et al., Antigen-specific transfer of functional programmed death ligand 1 from human APCs onto CD8+ T cells via trogocytosis. J Immunol 188, 744-752 (2012).
61. S. Daubeuf et al., Preferential transfer of certain plasma membrane proteins onto T and B cells by trogocytosis. PLoS One 5, e8716 (2010).
62. Y. T. Bryceson et al., Functional analysis of human NK cells by flow cytometry. Methods Mol Biol 612, 335-352 (2010).
63. J. C. Siebert et al., An analytical workflow for investigating cytokine profiles. Cytometry A 73, 289-298 (2008).
64. G. Alter, J. M. Malenfant, M. Altfeld, CD107a as a functional marker for the identification of natural killer cell activity. Joumal of immunological methods 294, 15-22 (2004).
65. S. Shabrish, M. Gupta, M. Madkaikar, A Modified NK Cell Degranulation Assay Applicable for Routine Evaluation of NK Cell Function. Joumal of immunology research 2016, U.S. Pat. No. 3,769,590 (2016).
66. L. Lamoreaux, M. Roederer, R. Koup, Intracellular cytokine optimization and standard operating procedure. Nature protocols 1, 1507-1516 (2006).
67. J. Yoneda et al., Expression of angiogenesis-related genes and progression of human ovarian carcinomas in nude mice. J Natl Cancer Inst 90, 447-454 (1998).
68. A. S. Cerdeira et al., Conversion of peripheral blood NK cells to a decidual NK-like phenotype by a cocktail of defined factors. J Immunol 190, 3939-3948 (2013).
69. H. D. Kopcow et al., Human decidual NK cells form immature activating synapses and are not cytotoxic. Proc Natl Acad Sci USA 102, 15563-15568 (2005).
70. A. Bruno et al., Angiogenin and the MMP9-TIMP2 axis are up-regulated in proangiogenic, decidual NK-like cells from patients with colorectal cancer. Faseb j 32, 5365-5377 (2018).
71. A. R. Venkitaraman, Linking the cellular functions of BRCA genes to cancer pathogenesis and treatment. Annual review of pathology 4, 461-487 (2009).
72. R. Vento-Tormo et al., Single-cell reconstruction of the early maternal-fetal interface in humans. Nature 563, 347-353 (2018).
73. L. Huergo-Zapico et al., NK-cell Editing Mediates Epithelial-to-Mesenchymal Transition via Phenotypic and Proteomic Changes in Melanoma Cell Lines. Cancer Res 78, 3913-3925 (2018).
74. A. Labani-Motlagh et al., Differential expression of ligands for NKG2D and DNAM-1 receptors by epithelial ovarian cancer-derived exosomes and its influence on NK cell cytotoxicity. Tumour Biol 37, 5455-5466 (2016).
75. S. Gasser, D. H. Raulet, The DNA damage response arouses the immune system. Cancer Res 66, 3959-3962 (2006).
76. S. Bugide, M. R. Green, N. Wajapeyee, Inhibition of Enhancer of zeste homolog 2 (EZH2) induces natural killer cell-mediated eradication of hepatocellular carcinoma cells. Proc Natl Acad Sci USA 115, E3509-e3518 (2018).
77. A. Yurtsever et al., Assessment of genetic markers and glioblastoma stem-like cells in activation of dendritic cells. Human cell 26, 105-113 (2013).
78. G. Belaaloui et al., Functional characterization of human CD34+ cells that express low or high levels of the membrane antigen CD111 (nectin 1). Leukemia 17, 1137-1145 (2003).
79. M. S. Derycke et al., Nectin 4 overexpression in ovarian cancer tissues and serum: potential role as a serum biomarker. Am J Clin Pathol 134, 835-845 (2010).
80. G. Damia, M. Broggini, Platinum Resistance in Ovarian Cancer Role of DNA Repair. Cancers 11, (2019).
81. D. Hudrisier, P. Bongrand, Intercellular transfer of antigen-presenting cell determinants onto T cells: molecular mechanisms and biological significance. Faseb j 16, 477-486 (2002).
82. B. J. Quah et al., Bystander B cells rapidly acquire antigen receptors from activated B cells by membrane transfer. Proc Nati Acad Sci USA 105, 4259-4264 (2008).
83. K. Miyake et al., Trogocytosis of peptide-MHC class II complexes from dendritic cells confers antigen-presenting ability on basophils. Proc Natl Acad Sci USA 114, 1111-1116 (2017).
84. F. E. McCann, P. Eissmann, B. Onfelt, R. Leung, D. M. Davis, The activating NKG2D ligand MHC class I-related chain A transfers from target cells to NK cells in a manner that allows functional consequences. J Immunol 178, 3418-3426 (2007).
85. J. Tabiasco et al., Active trans-synaptic capture of membrane fragments by natural killer cells. Eur J Immunol 32, 1502-1508 (2002).
86. C. I. Domaica et al., Tumour-experienced T cells promote NK cell activity through trogocytosis of NKG2D and NKp46 ligands. EMBO Rep 10, 908-915 (2009).
87. T. Tilburgs, J. H. Evans, A. C. Crespo, J. L. Strominger, The HLA-G cycle provides for both NK tolerance and immunity at the maternal-fetal interface. Proc Nati Acad Sci USA 112, 13312-13317 (2015).
88. J. Caumartin et al., Trogocytosis-based generation of suppressive NK cells. Embo j 26, 1423-1433 (2007).
89. M. Hamieh et al., CAR T cell trogocytosis and cooperative killing regulate tumour antigen escape. Nature 568, 112-116 (2019).
90. M. Carlsten et al., Primary human tumor cells expressing CD155 impair tumor targeting by down-regulating DNAM-1 on NK cells. J Immunol 183, 4921-4930 (2009).
91. J. A. Belisle et al., Peritoneal natural killer cells from epithelial ovarian cancer patients show an altered phenotype and bind to the tumour marker MUC16 (CA125). Immunology 122, 418-429 (2007).
92. M. D. Gutierrez-Lopez et al., The sheddase activity of ADAM17/TACE is regulated by the tetraspanin CD9. Cellular and molecular life sciences: CMLS 68, 3275-3292 (2011).
93. L. L. Lanier, NKG2D Receptor and Its Ligands in Host Defense. Cancer immunology research 3, 575-582 (2015).
94. G. Okumura et al., Tumor-derived soluble CD155 inhibits DNAM-1-mediated antitumor activity of natural killer cells. J Exp Med 217, (2020).
95. M. Vitale, C. Cantoni, G. Pietra, M. C. Mingari, L. Moretta, Effect of tumor cells and tumor microenvironment on NK-cell function. Eur J Immunol 44, 1582-1592 (2014).
96. A. W. Zhang et al., Interfaces of Malignant and Immunologic Clonal Dynamics in Ovarian Cancer. Cell 173, 1755-1769.e1722 (2018).
97. D. W. Garsed et al., Homologous Recombination DNA Repair Pathway Disruption and Retinoblastoma Protein Loss Are Associated with Exceptional Survival in High-Grade Serous Ovarian Cancer. Clin Cancer Res 24, 569-580 (2018).
98. L. G. Meza Guzman, N. Keating, S. E. Nicholson, Natural Killer Cells: Tumor Surveillance and Signaling. Cancers 12, (2020).
99. D. L. Hermanson et al., Induced Pluripotent Stem Cell-Derived Natural Killer Cells for Treatment of Ovarian Cancer. Stem Cells 34, 93-101 (2016).
100. A. Tremblay-McLean, S. Coenraads, Z. Kiani, F. P. Dupuy, N. F. Bernard, Expression of ligands for activating natural killer cell receptors on cell lines commonly used to assess natural killer cell function. BMC immunology 20, 8 (2019).
101. G. Han et al., Atomic mass tag of bismuth-209 for increasing the immunoassay multiplexing capacity of mass cytometry. Cytometry A 91, 1150-1163 (2017).
102. S. C. Bendall et al., Single-cell mass cytometry of differential immune and drug responses across a human hematopoietic continuum. Science 332, 687-696 (2011).
103. S. Lorenzo-Herrero, C. Sordo-Bahamonde, S. Gonzalez, A. Lopez-Soto, Evaluation of NK cell cytotoxic activity against malignant cells by the calcein assay. Methods Enzymol 631, 483-495 (2020).
104. N. S. Ramadoss et al., Enhancing natural killer cell function with gp41-targeting bispecific antibodies to combat HIV infection. bioRxiv, 760280 (2019).
105. N. Kotecha, P. O. Krutzik, J. M. Irish, Web-based analysis and publication of flow cytometry experiments. Current protocols in cytometry/editorial board, J. Paul Robinson, managing editor . . . [et al.] Chapter 10, Unit10 17 (2010).

What is claimed is:

1. A method for treating an individual with cancer with NK cell immunotherapy, the method comprising:
evaluating CD9 expression within the cancer microenvironment for an individual eligible to receive NK cell immunotherapy;
determining the level of CD9 positivity within the cancer;
administering NK cell immunotherapy the individual in combination with an effective dose of a CD9 or trogocytosis blocking agent if the CD9 positivity of the cancer is high.

2. The method of claim 1, wherein the cancer is a solid cancer.

3. The method of claim 1 or claim 2, wherein the cancer is ovarian cancer.

4. The method of claim 3, wherein the ovarian cancer is high grade serous cancer.

5. The method of any of claims 1-4, wherein the CD9 blocking agent is an antibody that binds to CD9.

6. The method of any of claims 1-4, wherein the CD9 blocking agent is administered prior to NK cell immunotherapy.

7. The method of claim 6, wherein the CD9 blocking agent is administered intra-tumorally.

8. The method of any of claims 1-4, wherein NK cells are pre-treated prior to administration with agent that inhibits trogocytosis.

9. The method of any of claims 1-8, wherein the NK cell immunotherapy comprises administering an effective dose of an in vitro expanded autologous or allogeneic NK cell population.

10. The method of any of claims 1-8, wherein the NK cell immunotherapy comprises administering an effective dose of an in vitro expanded human NK cell line.

11. The method of claim 9 or claim 10, wherein the NK cells are genetically modified prior to administration.

12. The method of any of claims 1-7, wherein the NK cell immunotherapy comprises administering an agent that activates endogenous NK cells.

13. A prognostic method of predicting a poor prognosis for a patient having ovarian cancer and treating the patient for the ovarian cancer, the method comprising:
obtaining a sample of ovarian tumor tissue from the patient, wherein the ovarian tumor tissue comprises a population of infiltrating NK cells;
measuring frequency of decidual-like NK cells in the population of infiltrating NK cells, wherein increased frequency of decidual-like NK cells compared to reference value ranges for a control population of NK cells indicates that the patient has a poor prognosis; and
treating the patient with surgery, radiation therapy, chemotherapy, targeted therapy, anti-angiogenic therapy, or immunotherapy, or any combination thereof, if the patient is identified as having a poor prognosis.

14. The method of claim 13, wherein said measuring the frequency of decidual-like NK cells comprises detecting at least one NK cell expressing a CD9 marker, wherein expression of the CD9 marker indicates that the NK cell is a decidual-like NK cell.

15. The method of claim 14, further comprising detecting at least one decidual-like NK cell expressing a CD9 marker in combination with one or more additional markers selected from the group consisting of CD56 and chemokine receptor CXCR3.

16. The method of any of claims 13-15, wherein the frequency of the decidual-like NK cells in the population of infiltrating NK cells is at least 29%.

17. The method of claim 16, wherein the frequency of the decidual-like NK cells in the population of infiltrating NK cells is at least 60%.

18. The method of any of claims 13-17, further comprising measuring levels of expression of one or more activating NK receptor ligands on cancerous cells in the sample of ovarian tumor tissue, wherein increased frequency of the decidual-like NK cells in combination with decreased levels of expression of one or more activating NK receptor ligands compared to the levels of expression of said NK receptor ligands on control ovarian cells indicates that the patient has a poor prognosis.

19. The method of any of claims 13-18, further comprising measuring levels of expression of one or more inhibitory NK receptor ligands on cancerous cells in the sample of ovarian tumor tissue, wherein increased frequency of the decidual-like NK cells in combination with increased levels of expression of one or more inhibitory NK receptor ligands compared to the levels of expression of said NK receptor ligands on control ovarian cells indicates that the patient has a poor prognosis.

20. The method of claim 18 or 19, wherein levels of NK receptor ligands are measured in ovarian cancer cells expressing E-cadherin (E tumor compartment), ovarian cancer cells coexpressing E-cadherin and vimentin (EV tumor compartment), and ovarian cancer cells expressing vimentin (V tumor compartment).

21. The method of any of claims 19-21, further comprising administering NK cellular immunotherapy to the patient if activating NK receptor ligands are detected on the ovarian cancer cells and increased levels of expression of the one or more inhibitory NK receptor ligands are not detected on the ovarian cancer cells.

22. The method of any of claims 13-21, further comprising measuring frequency of NK cells in the population of infiltrating NK cells producing at least three cytokines selected from the group consisting of IL-8, IL-10, TNFα, and IFNγ, wherein increased frequency of the decidual-like NK cells in combination with decreased frequency of the NK cells producing at least three cytokines selected from the group consisting of IL-8, IL-10, TNFα, and IFNγ indicates that the patient has a poor prognosis.

23. The method of any of claims 13-22, further comprising measuring levels of perforin and granzyme B produced by the population of infiltrating NK cells, wherein increased frequency of the decidual-like NK cells in combination with decreased levels of the perforin and the granzyme B compared to the levels of the perforin and the levels of the granzyme B for a control population of NK cells indicate that the patient has a poor prognosis.

25. The method of any of claims 13-24, wherein the ovarian cancer is high-grade serous ovarian cancer.

26. The method of any of claims 13-25, wherein the sample of ovarian tumor tissue is a biopsy or surgical specimen.

27. The method of any of claims 13-26, wherein said measuring the frequency of decidual-like NK cells in the population of infiltrating NK cells comprises performing flow cytometry, cytometry by time of flight (CyTOF), immunohistochemistry, immunofluorescence, CO-detection by indexing (CODEX), multiplexed ion beam imaging (MIBI), or other multi-parametric single cell analysis technology.

28. The method of any of claims 13-27, wherein the NK cellular immunotherapy comprises administration of one or more cytokines that activate NK cells to the patient, adoptive transfer of NK cells to the patient, or a combination thereof.

29. The method of any of claims 13-28, wherein the patient is a human being.

30. A method of predicting whether a patient having ovarian cancer will benefit from natural killer (NK) cellular immunotherapy and treating the patient for the ovarian cancer, the method comprising:
obtaining a sample of ovarian tumor tissue from the patient;
measuring NK receptor ligand distribution on cancerous cells in the ovarian tumor tissue, wherein detection of one or more activating NK receptor ligands indicates that the patient will benefit from NK cellular immunotherapy, and detection of one or more inhibitory NK receptor ligands indicates that the patient will not benefit from NK cellular immunotherapy; and
administering NK cellular immunotherapy to the patient if the NK receptor ligand distribution indicates that the patient will benefit from NK cell immunotherapy.

31. The method of any of claim 30, wherein the method is performed prior to treatment of the patient with the NK cellular immunotherapy.

32. The method of claim 17, wherein the patient is undergoing immunotherapy.

33. The method of any of claims 30-32, wherein the NK cellular immunotherapy comprises administration of one or more cytokines that activate NK cells to the patient, adoptive transfer of NK cells to the patient, or a combination thereof.

34. The method of any of claims 30-33, wherein administering NK cellular immunotherapy comprises administering engineered NK cells comprising an NK activating receptor.

35. The method of any of claims 30-34, wherein the activating NK receptor ligands activate the NKG2D receptor.

36. The method of claim 35, wherein the activating NK receptor ligands are selected from the group consisting of ULBP1, ULBP2, ULPBP3, ULPBP4, ULBP5, ULBP6, and MICA/B.

37. The method of any of claims 30-36, further comprising measuring frequency of NK cells in a population of infiltrating NK cells producing at least three cytokines selected from the group consisting of IL-8, IL-10, TNFα, GM-CSF and IFNγ in the sample of ovarian tumor tissue, wherein decreased frequency of the NK cells producing at least three cytokines selected from the group consisting of IL-8, IL-10, TNFα, and IFNγ indicates that the patient will not benefit from NK cell immunotherapy.

38. The method of any of claims 30-37, further comprising measuring levels of perforin and granzyme B produced by a population of infiltrating NK cells in the sample of ovarian tumor tissue, wherein decreased levels of the perforin and the granzyme B compared to the levels of the perforin and the levels of the granzyme B for a control population of NK cells indicate that the patient will not benefit from NK cell immunotherapy.
